SEQUENCE LISTING (85 sequences)

SEQ ID NO: 1 - DNA, 576 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggc

SEQ ID NO: 2 - DNA, 575 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcggc

SEQ ID NO: 3 - DNA, 626 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 4 - DNA, 1849 bp, Homo sapiens
cgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggtttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaaaaaaaaaaaaaaaaaaaaaaaaaaaaaaaaaaaaaaaaaaaaaaaaaaaaaa

SEQ ID NO: 5 - DNA, 1938 bp, Homo sapiens
gccagctctcgcactctgttcttccgccgctccgccgtcgcgtttctctgccggtcgcaatggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccccggaggagcacccagtgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaaccaaaaaaaaaaaaaaaaaaaaaaaaaaaaaaaaaa

SEQ ID NO: 6 - DNA, 4509 bp, Homo sapiens
agattgctcatgtaactcttgagtttacatgtaatcaacatatgctcattgaaaacgggattgcttcaagaggactttgagtccagggtgattaggtaagtaaaagatgtaaaaaggtagaaaatttttgtcacttgagtctaaataattgttcttataagtgccaacgcctgtttctgttaggctcagaagatcaaaggatttggctcttttaaaatatagaaagctctagcttcagctagaatttaggcctttagtaatagccctaatttttatgaagccattttgttccagtgatcttttggtgagagatgctatgtaagtactattcttcagaattaggtgtctttttaccctaatgaaataatttagattgcttttgatacaggtaaaacaaatatcctggcttccataattgtagaaaaaacttcatataggaatccttgttgtatcaaagtagcacctgatgggaatgaacagacaggaatggatgaaggatagcagtttgcgttccatttcaagcctatgggctcacacatttattcagataagaacaccacctttcactagataaactccaacagtattcatgcatacttttgaatggcatgtaggaaatgtttgataggtacataatgtattcacttcaggtcactaatgtaatacggggtcgtgctccttagtgttgacagatcacctatggttctccaaaatgaacattctagtacaggaggtctagggaggaacctgagagtatactaatgcctaggaactttctctggagtggcaagagcagtgggaagaattatgtcaatagctacagaaataagggagtaagaacaagtcatctctctagtgaattcttcttcactttactgagataaacatacatgttaatgagcttgagttttcccaaaagtataattcttctggttcttctaagaaaatggcactccctggaaacaaggaagaaccaaatttattcgcctttgtagcagttgggaaagttagtgctaggaagtcttattgatttatagtaggctttaatctggatattgctggtaaagtttattctaaaacctgaactctggataagtaatacaaaaagcttctcaaccttccaagcaaaattgagagctttcaggttatgtgagtaatttggtctcttgggtgcttaattcattccttgaagctcatttttgtgatctcttccaagattgcatttgcttggaggtagggagttagacaagatggtatgaggtccctaaattttgactttccaagcaaaattggacagtggttcctaaattgctaacatcctcgtttcttcctaaggcttctcatgtttcatatatagtagccttcccaaaatcccatttcccacccccccccccccaacccatgtagagagaacgaacctgtctcccttcctgtacagagtacgggatccttcaactttcacacaggctgcagtgtctgccacacatttagctcaacttttttttagccttaaagtgatgtccgctgcatctgtcgctgggttgcaccttgtggatttagtttgcataaattttctcagcttaaacaaagttaacattgaatagagtaagcttaccataaagggcttaataaatgccatgcatgtctacattcggtgtggaaattgagctagtcaggttgatatttaacattgtaggttctttgttaatttatatgaaataatggttatcatttaactcttcaggttagctttgtacatagcatctcactttgcacaacaaccctgcaaggtaagtattgttattcttgtgctacaaatgaagttgactgagaggaggagtaccacgtccaaggtcacacagctattaaatggcagggctgggatactggcctgtgactcagaacttgatgctttccccccacgccacgcatgccaggttgcccttcctttcagaaatggtggaagtcctgcaaaatgcaataaactgaagtaatgtagcttctattaatacaaagtaaataactcagatttactggattttaaaccttattccttgggtaaacaatctgtgactgacttcacaccaaatatttgttggcggaggatttggactttagggataaaagtggatacattttttattttacaaactctgtatttgaacttaattattggctcttcaattttacgttaccagctttttttttttttttttttaatgaatttgatttacatcatggtcaaacaaaaattgttgagcagggaaaataaactactttctggattccttcttgaattttctcatgtgccctagagaaaatgtgttccacattaaggtgttactttttccaggggtgtgttcatttaaaaagaatgaagccaggcaatgtttatttttcttttacctataaataaatgaatggattaatcattgtatacttgactcccatgttggtagggattttagataggaggctatttcttgtctgtgcttctcaataccccataagcagttgcttcatggatgtatatactaataagcagtgaaagaaagtgcatgttcaaagaatacaacaaggagtctggatattttgcaatcatctttatatattacggtgctctgaattaaaagctaaaagttactgggtatgtctgacaccttagtgctttatctttgttctactaattttctgtgccccaatcccacttaaccctagcctcattccttatctgtaagataggggataataccactgtaaggttattattaagattgaataaggataaaatttataatgggttttagcaaatggcagaaaatattttctgaagaaaaccaagtgctattaaaaaaacatcacaagccttgggcttactttgggattttaaaaaccaagagaaaatggatggctgaactttcaaacatttggtaaatattatagtattgtagttcagagctctggattctttgcattttgcctgctgggtgagaaggaataaaagtttgtgcctttttttttttttaatcactttaatttcaaaacaatgtgtttaaccatttgtgggagtaattttcattttgtgagcctgaagcattttgattcagtgggaatttctggtgatttatatctggaatagaagtgagcttaagtttagctattctaacgttgaaaaaggaagcaatgtttctattggattctaaagtatattttcaaaaatattctgaagtatttgtatatcttaaacttggagttaagacagcttagctttgaagataagagaaactagatgtgtgcattttctatccagatgtgtttgttgctggaactaaatgaaacagtacatggtaacccttgaaaggttttaaacttgtttctgtaactgctaatctacatactctcaagtcactaaccttcctctttgatctctttgtaggctgaccaactgactgaagagcagattgcagaattcaaagaagctttttcactatttgacaaagatggtgatggaactataacaacaaaggaattgggaactgtaatgagatctcttgggcagaatcccacagaagcagagttacaggacatgattaatgaagtagatgctgatggtaatggcacaattgacttccctgaatttctgacaatgatggcaagaaaaatgaaagacacagacagtgaagaagaaattagagaagcattccgtgtgtttgataaggatggcaatggctatattagtgctgcagaacttcgccatgtgatgacaaaccttggagagaagttaacagatgaagaagttgatgaaatgatcagggaagcagatattgatggtgatggtcaagtaaactatgaagagtttgtacaaatgatgacagcaaagtgaagaccttgtacagaatgtgttaaatttcttgtacaaaattgtttatttgccttttctttgtttgtaacttatctgtaaaaggtttctccctactgtcaaaaaaatatgcatgtatagtaattaggacttcattcctccatgttttcttcccttatcttactgtcattgtcctaaaaccttattttagaaaattgatcaagtaacatgttgcatgtggcttactctggatatatctaagcccttctgcacatctaaacttagatggagttggtcaaatgagggaacatctgggttatgcattttttaaagtagttttctttaggaactgtcagcatgttgttgttgaagtgtggagttgtaactctgcgtggactatggacagtcaacaatatgtacttaaaagttgcactattgcaaaacgggtgtattatccaggtactcgtacactatttttttgtactgctggtcctgtaccagaaacattttcttttattgttacttgctttttaaactttgtttagccacttaaaatctgcttatggcacaatttgcctcaaaatccattccaagttgtatatttgttttccaataaaaaaattacaatttacacaaaaaaaaaaaaaaaa

SEQ ID NO: 7 - DNA, 1077 bp, Homo sapiens
gcggctgcagcgctctcgtcttctgcggctctcggtgccctctccttttcgtttccggaaacatggcctccggtgtggctgtctctgatggtgtcatcaaggtgttcaacgacatgaaggtgcgtaagtcttcaacgccagaggaggtgaagaagcgcaagaaggcggtgctcttctgcctgagtgaggacaagaagaacatcatcctggaggagggcaaggagatcctggtgggcgatgtgggccagactgtcgacgatccctacgccacctttgtcaagatgctgccagataaggactgccgctatgccctctatgatgcaacctatgagaccaaggagagcaagaaggaggatctggtgtttatcttctgggcccccgagtctgcgccccttaagagcaaaatgatttatgccagctccaaggacgccatcaagaagaagctgacagggatcaagcatgaattgcaagcaaactgctacgaggaggtcaaggaccgctgcaccctggcagagaagctggggggcagtgccgtcatctccctggagggcaagcctttgtgagccccttctggccccctgcctggagcatctggcagccccacacctgcccttgggggttgcaggctgcccccttcctgccagaccggaggggctggggggatcccagcagggggagggcaatcccttcaccccagttgccaaacagaccccccaccccctggattttccttctccctccatcccttgacggttctggccttcccaaactgcttttgatcttttgattcctcttgggctgaagcagaccaagttccccccaggcaccccagttgtgggggagcctgtattttttttaacaacatccccattccccacctggtcctcccccttcccatgctgccaacttctaaccgcaatagtgactctgtgcttgtctgtttagttctgtgtataaatggaatgttgtggagatgacccctccctgtgccggctggttcctctcccttttcccctggtcacggctactcatggaagcaggaccagtaagggaccttcgaaaaaaaaaaaaaaaaaa

SEQ ID NO: 8 - DNA, 1652 bp, Homo sapiens
cagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaaaaaaaaaaaaaaa

SEQ ID NO: 9 - DNA, 1426 bp, Homo sapiens
cttttctttgcggaatcaccatggcggctgggaccctgtacacgtatcctgaaaactggagggccttcaaggctctcatcgctgctcagtacagcggggctcaggtccgcgtgctctccgcaccaccccacttccattttggccaaaccaaccgcacccctgaatttctccgcaaatttcctgccggcaaggtcccagcatttgagggtgatgatggattctgtgtgtttgagagcaacgccattgcctactatgtgagcaatgaggagctgcggggaagtactccagaggcagcagcccaggtggtgcagtgggtgagctttgctgattccgatatagtgcccccagccagtacctgggtgttccccaccttgggcatcatgcaccacaacaaacaggccactgagaatgcaaaggaggaagtgaggcgaattctggggctgctggatgcttacttgaagacgaggacttttctggtgggcgaacgagtgacattggctgacatcacagttgtctgcaccctgttgtggctctataagcaggttctagagccttctttccgccaggcctttcccaataccaaccgctggttcctcacctgcattaaccagccccagttccgggctgtcttgggcgaagtgaaactgtgtgagaagatggcccagtttgatgctaaaaagtttgcagagacccaacctaaaaaggacacaccacggaaagagaagggttcacgggaagagaagcagaagccccaggctgagcggaaggaggagaaaaaggcggctgcccctgctcctgaggaggagatggatgaatgtgagcaggcgctggctgctgagcccaaggccaaggaccccttcgctcacctgcccaagagtacctttgtgttggatgaatttaagcgcaagtactccaatgaggacacactctctgtggcactgccatatttctgggagcactttgataaggacggctggtccctgtggtactcagagtatcgcttccctgaagaactcactcagaccttcatgagctgcaatctcatcactggaatgttccagcgactggacaagctgaggaagaatgccttcgccagtgtcatcctttttggaaccaacaatagcagctccatttctggagtctgggtcttccgaggccaggagcttgcctttccgctgagtccagattggcaggtggactacgagtcatacacatggcggaaactggatcctggcagcgaggagacccagacgctggttcgagagtacttttcctgggagggggccttccagcatgtgggcaaagccttcaatcagggcaagatcttcaagtgaacatctctcgccatcacctagctgcctgcacctgcccttcagggagatgggggtcattaaaggaaactgaacattgaaaaaaaaaaaaaaaa

SEQ ID NO: 10 - DNA, 924 bp, Homo sapiens
gagagtcgtcggggtttcctgcttcaacagtgcttggacggaacccggcgctcgttccccaccccggccggccgcccatagccagccctccgtcacctcttcaccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtacccaaaaaaaaaaaaaaaaaaa

SEQ ID NO: 11 - DNA, 1428 bp, Homo sapiens
ggcggttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaaggtaactagtgtgtgggtaatggactacatctccaagcaggccgtgcgcgcgaggagccttgatttgagggcgtaggtgtcgcgtgggcttctgggagattgagttcggtcttgtgagccctcttaaccgctggaaatagaggcgcacctcgtgcagtgcccacaacacgcggcagtccacaccgctgcgtggtcttagggacgtatagctgtaagagctaggacagggtgcggagagtgataaatacaagctgtcacatgtctttgtggcctgggcctctgacccccaacgactcttgggaaatgtaggtttagttctatgtgccgagtgtgtgtattctgagccatttctcccttctatatagaagccagctgaagatgagtggggtaaaaccccagacgccatgaaagctgccatggccctggagaaaaagctgaaccaggcccttttggatcttcatgccctgggttctgcccgcacggacccccatgtacgtacccgctgcatccatggctacccaaccatacccctcaagcctctgctccctttgggcaaatttccttcagagcctcatttcacacctgtcacattttaatctgcaactggctgctctctccccctcttttccagggattgggtttctaatttctccctcttctctctcagctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaactatcctaacaagccttggaccaaatggaaataaagctttttgatgcgaaaaaaaaaaaaaaaa

SEQ ID NO: 12 - DNA, 1290 bp, Homo sapiens
gtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaaaaaaaaaaaaaaaaa

SEQ ID NO: 13 - DNA, 1551 bp, Homo sapiens
ccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagacagtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaag

SEQ ID NO: 14 - DNA, 840 bp, Homo sapiens
cccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcaaaaaaaaaaaaaaa

SEQ ID NO: 15 - DNA, 1771 bp, Homo sapiens
ggcggccaggccgggcgcggagtgggcgcgcggggccggaggaggggccagcgaccgcggcaccgcctgtgcccgcccgcccctccgcagccgctacttaagaggctccagcgccggccccgccctagtgcgttacttacctcgactcttagcttgtcggggacggtaaccgggacccggtgtctgctcctgtcgccttcgcctcctaatccctagccactatgcgtgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattgatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactctgattgtgccttcatggtagacaatgaggccatctatgacatctgtcgtagaaacctcgatatcgagcgcccaacctacactaaccttaaccgccttattagccagattgtgtcctccatcactgcttccctgagatttgatggagccctgaatgttgacctgacagaattccagaccaacctggtgccctacccccgcatccacttccctctggccacatatgcccctgtcatctctgctgagaaagcctaccatgaacagctttctgtagcagagatcaccaatgcttgctttgagccagccaaccagatggtgaaatgtgaccctcgccatggtaaatacatggcttgctgcctgttgtaccgtggtgacgtggttcccaaagatgtcaatgctgccattgccaccatcaaaaccaagcgcagcatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttcagcttaactgacagacgttaaagctttctggttagattgttttcacttggtgatcatgtcttttccatgtgtacctgtaatatttttccatcatatctcaaagtaaagtcattaacatcaaaaaaaaaaaaaaaaaaaaaa

SEQ ID NO: 16 - DNA, 840 bp, Homo sapiens; identical to SEQ ID NO: 14

SEQ ID NO: 17 - DNA, 858 bp, Homo sapiens
cgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcaaaaaaaaaaaaaaaaaaaaaaaaaaa

SEQ ID NO: 18 - DNA, 3227 bp, Homo sapiens
cgactccttagagcatggcatggctcagaggtgctggtaaaactgatgggggtttttgctgtccctcccctcagctccgacaccatgtggatccaggttcggaccatggatgggaggcagacccacacggtggactcgctgtccaggctgaccaaggtggaggagctgaggcggaagatccaggagctgttccacgtggagccaggcctgcagaggctgttctacaggggcaaacagatggaggacggccataccctcttcgactacgaggtccgcctgaatgacaccatccagctcctggtccgccagagcctcgtgctcccccacagcaccaaggagcgggactccgagctctccgacaccgactccggctgctgcctgggccagagtgagtcagacaagtcctccacccacggtgaggcggccgccgagactgacagcaggccagccgatgaggacatgtgggatgagacggaattggggctgtacaaggtcaatgagtacgtcgatgctcgggacacgaacatgggggcgtggtttgaggcgcaggtggtcagggtgacgcggaaggccccctcccgggacgagccctgcagctccacgtccaggccggcgctggaggaggacgtcatttaccacgtgaaatacgacgactacccggagaacggcgtggtccagatgaactccagggacgtccgagcgcgcgcccgcaccatcatcaagtggcaggacctggaggtgggccaggtggtcatgctcaactacaaccccgacaaccccaaggagcggggcttctggtacgacgcggagatctccaggaagcgcgagaccaggacggcgcgggaactctacgccaacgtggtgctgggggatgattctctgaacgactgtcggatcatcttcgtggacgaagtcttcaagattgagcggccgggtgaagggagccccatggttgacaaccccatgagacggaagagcgggccgtcctgcaagcactgcaaggacgacgtgaacagactctgccgggtctgcgcctgccacctgtgcgggggccggcaggaccccgacaagcagctcatgtgcgatgagtgcgacatggccttccacatctactgcctggacccgcccctcagcagtgttcccagcgaggacgagtggtactgccctgagtgccggaatgatgccagcgaggtggtactggcgggagagcggctgagagagagcaagaagaaggcgaagatggcctcggccacatcgtcctcacagcgggactggggcaagggcatggcctgtgtgggccgcaccaaggaatgtaccatcgtcccgtccaaccactacggacccatcccggggatccccgtgggcaccatgtggcggttccgagtccaggtcagcgagtcgggtgtccatcggccccacgtggctggcatacacggccggagcaacgacggagcgtactccctagtcctggcggggggctatgaggatgacgtggaccatgggaattttttcacatacacgggtagtggtggtcgagatctttccggcaacaagaggaccgcggaacagtcttgtgatcagaaactcaccaacaccaacagggcgctggctctcaactgctttgctcccatcaatgaccaagaaggggccgaggccaaggactggcggtcggggaagccggtcagggtggtgcgcaatgtcaagggtggcaagaatagcaagtacgcccccgctgagggcaaccgctatgatggcatctacaaggttgtgaaatactggcccgagaaggggaagtccgggtttctcgtgtggcgctaccttctgcggagggacgatgatgagcctggcccttggacgaaggaggggaaggaccggatcaagaagctggggctgaccatgcagtatccagaaggctacctggaagccctggccaaccgagagcgagagaaggagaacagcaagagggaggaggaggagcagcaggaggggggcttcgcgtcccccaggacgggcaagggcaagtggaagcggaagtcggcaggaggtggcccgagcagggccgggtccccgcgccggacatccaagaaaaccaaggtggagccctacagtctcacggcccagcagagcagcctcatcagagaggacaagagcaacgccaagctgtggaatgaggtcctggcgtcactcaaggaccggccggcgagcggcagcccgttccagttgttcctgagtaaagtggaggagacgttccagtgtatctgctgtcaggagctggtgttccggcccatcacgaccgtgtgccagcacaacgtgtgcaaggactgcctggacagatcctttcgggcacaggtgttcagctgccctgcctgccgctacgacctgggccgcagctatgccatgcaggtgaaccagcctctgcagaccgtcctcaaccagctcttccccggctacggcaatggccggtgatctccaagcacttctcgacaggcgttttgctgaaaacgtgtcggagggctcgttcatcggcactgattttgttcttagtgggcttaacttaaacaggtagtgtttcctccgttccctaaaaaggtttgtcttccttttttttttatttttatttttcaaatctatacattttcaggaatttatgtattctggctaaaagttggacttctcagtattgtgtttagttctttgaaaacataaaagcctgcaatttctcgacaaaacaacacaagattttttaaagatggaatcagaaactacgtggtgtggaggctgttgatgtttctggtgtcaagttctcagaagttgctgccaccaactctttaagaaggcgacaggatcagtccttctctcgggttctggcccccaaggtcagagcaagcatcttcctgacagcattttgtcatctaaagtccagtgacatggttccccgtggtggcccgtggcagcccgtggcatggcgtggctcagctgtctgttgaagttgttgcaaggaaaagaggaaacatctcgggcctagttcaaacctttgcctcaaagccatcccccaccagactgcttagcgtctgagatccgcgtgaaaagtcctctgcccacgagagcagggagttggggccacgcagaaatggcctcaaggggactctgctccacgtggggccaggcgtgtgactgacgctgtccgacgaaggcggccacggacggacgccagcacacgaagtcac

SEQ ID NO: 19 - DNA, 24 bp, Homo sapiens
ctccagggcctccgcaccatactc

SEQ ID NO: 20 - DNA, 24 bp, Homo sapiens
tggtggtggggaaggacaggaaca

SEQ ID NO: 21 - DNA, 24 bp, Homo sapiens
ggtcgaagtgcgggaagtaggtct

SEQ ID NO: 22 - DNA, 23 bp, Homo sapiens
gtcagcgcgtcggccaccttctt

SEQ ID NO: 23 - DNA, 24 bp, Homo sapiens
gccgcccactcagactttattcaa

SEQ ID NO: 24 - DNA, 22 bp, Homo sapiens
ccacagggcagtaacggcagac

SEQ ID NO: 25 - DNA, 25 bp, Homo sapiens
cataacagcatcaggagtggacaga

SEQ ID NO: 26 - DNA, 24 bp, Homo sapiens
ccatcactaaaggcaccgagcact

SEQ ID NO: 27 - DNA, 24 bp, Homo sapiens
cattagccacaccagccaccactt

SEQ ID NO: 28 - DNA, 24 bp, Homo sapiens
ggcccttcataatatcccccagtt

SEQ ID NO: 29 - DNA, 1289 bp, Homo sapiens
gtctgacgggcgatggcgcagccaatagacaggagcgctatccgcggtttctgattggctactttgttcgcattataaaaggcacgcgcgggcgcgaggcccttctctcgccaggcgtcctcgtggaaggcccgggaccgcgggatgggtgtcggcgtgaccaggcctgagctccctgtctctcctcagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 30 - DNA, 437 bp, Homo sapiens
cctttcctcagctgccgccaaggtgctcggtccttccgaggaagctaaggctgcgttggggtgaggccctcacttcatccggcgactagcaccgcgtccggcagcgccagccctacactcgcccgcgccatggcctctgtctccgagctcgcctgcatctactcggccctcattctgcacgacgatgaggtgacagtcacggccctggccaacgtcaacattgggagcctcatctgcaatgtaggggccggtggacctgctccagcagctggtgctgcaccagcaggaggtcctgccccctccactgctgctgctccagctgaggagaagaaagtggaagcaaagaaagaagaatccgaggagtctgatgatgacatgggctttggtctttttgactaaacctcttttataacatgttcaataaaaagctgaacttt

SEQ ID NO: 31 - DNA, 948 bp, Homo sapiens
caaaacaccaaatggcggatgacgccggtgcagcgggggggcccggaggccctggtggccctgggatggggaaccgcggtggcttccgcggaggtttcggcagtggcattcggggccggggtcgcggccgtggacggggccggggccgaggccgcggagctcgcggaggcaaggccgaggataaggagtggatgcccgtcaccaagttgggccgcttggtcaaggacatgaagatcaagtccctggaggagatctatctcttctccctgcccattaaggaatcagagatcattgatttcttcctgggggcctctctcaaggatgaggttttgaagattatgccagtgcagaagcagacccgtgccggccagcgcaccaggttcaaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtgacaggccgctgcggctctgtgctggtacgcctcatccctgcacccaggggcactggcatcgtctccgcacctgtgcctaagaagctgctcatgatggctggtatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaattaagcgtgaaaaaaaaaaaaaaaaaa

SEQ ID NO: 32 - DNA, 921 bp, Homo sapiens
cgcgactcccacttccgcccttttggctctctgaccagcaccatggcggttggcaagaacaagcgccttacgaaaggcggcaaaaagggagccaagaagaaagtggttgatccattttctaagaaagattggtatgatgtgaaagcacctgctatgttcaatataagaaatattggaaagacgctcgtcaccaggacccaaggaaccaaaattgcatctgatggtctcaagggtcgtgtgtttgaagtgagtcttgctgatttgcagaatgatgaagttgcatttagaaaattcaagctgattactgaagatgttcagggtaaaaactgcctgactaacttccatggcatggatcttacccgtgacaaaatgtgttccatggtcaaaaaatggcagacaatgattgaagctcacgttgatgtcaagactaccgatggttacttgcttcgtctgttctgtgttggttttactaaaaaacgcaacaatcagatacggaagacctcttatgctcagcaccaacaggtccgccaaatccggaagaagatgatggaaatcatgacccgagaggtgcagacaaatgacttgaaagaagtggtcaataaattgattccagacagcattggaaaagacatagaaaaggcttgccaatctatttatcctctccatgatgtcttcgttagaaaagtaaaaatgctgaagaagcccaagtttgaattgggaaagctcatggagcttcatggtgaaggcagtagttctggaaaagccactggggacgagacaggtgctaaagttgaacgagctgatggatatgaaccaccagtccaagaatctgtttaaagttcagacttcaaatagtggcaaataaaaagtgctatttgtgatggtttgcttctgaaaaaaaaaaaaaaaaaaaaaaaaaaa

SEQ ID NO: 33 - DNA, 792 bp, Homo sapiens
atggcccggggccccaagaagcatctgaagcgggtggcagctccaaagcattggatgctggataaattgaccggtgtgtttgctcctcgtccatccaccggtccccacaagttgagagagtgtctccccctcatcattttcctgaggaacagacttaagtatgccctgacaggagatgaagtaaagaagatttgcatgcagcggttcattaaaatcgatggcaaggtccgaactgatataacctaccctgctggattcatggatgtcatcagcattgacaagacgggagagaatttccgtctgatctatgacaccaagggtcgctttgctgtacatcgtattacacctgaggaggccaagtacaagttgtgcaaagtgagaaagatctttgtgggcacaaaaggaatccctcatctggtgactcatgatgcccgcaccatccgctaccccgatcccctcatcaaggtgaatgataccattcagattgatttagagactggcaagattactgatttcatcaagttcgacactggtaacctgtgtatggtgactggaggtgctaacctaggaagaattggtgtgatcaccaacagagagaggcaccctggatcttttgacgtggttcacgtgaaagatgccaatggcaacagctttgccactcgactttccaacatttttgttattggcaagggcaacaaaccatggatttctcttccccgaggaaagggtatccgcctcaccattgctgaagagagagacaaaagactggctgccaaacagagcagtggctaa

SEQ ID NO: 34 - DNA, 845 bp, Homo sapiens
cctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctgaaaaaaaaaaaaaaaaaaaaaaaaaaaaaaaa

SEQ ID NO: 35 - DNA, 672 bp, Homo sapiens
gagagagagcgagagaactagtctcgagtttttttttttttttttttttttttttttttttttttttttttttccagccccggtaccggaccctgcagccgcagagatgttgatgcctaaaaaaaaccggattgccatttatgaactcctttttaaggagggagtcatggtggccaagaaggatgtccacatgcctaagcacccggagctggcagacaagaatgtgcccaaccttcatgtcatgaaggccatgcagtctctcaagtcccgaggctacgtgaaggaacagtttgcctggagacatttctactggtaccttaccaatgagggtatccagtatctccgtgattaccttcatctgcccccggagattgtgcctgccaccctacgccgtagccgtccagagactggcaggcctcggcctaaaggtctggagggtgagcgacctgcgagactcacaagaggggaagctgacagagatacctacagacggagtgctgtgccacctggtgccgacaagaaagccgaggctggggctgggtcagcaaccgaattccagtttagaggcggatttggtcgtggacgtggtcagccacctcagtaaaattggagaggattcttttgcattgaataaacttacagccaaaaaaccttaaaaaaaaaaaaaaaaaa

SEQ ID NO: 36 - DNA, 680 bp, Homo sapiens
ctgatgttggagcggccgcgataaggccatttttttttttttttttttttttttttttttttttttttttttttttttttttcttttcaggcggccgggaagatggcggacattcagactgagcgtgcctaccaaaagcagccgaccatctttcaaaacaagaagagggtcctgctgggagaaactggcaaggagaagctcccgcggtactacaagaacatcggtctgggcttcaagacacccaaggaggctattgagggcacctacattgacaagaaatgccccttcactggtaatgtgtccattcgagggcggatcctctctggcgtggtgaccaagatgaagatgcagaggaccattgtcatccgccgagactatctgcactacatccgcaagtacaaccgcttcgagaagcgccacaagaacatgtctgtacacctgtccccctgcttcagggacgtccagatcggtgacatcgtcacagtgggcgagtgccggcctctgagcaagacagtgcgcttcaacgtgctcaaggtcaccaaggctgccggcaccaagaagcagttccagaagttctgaggctggacatcggcccgctccccacaatgaaataaagttattttctcattcccaaaaaaaaaaaaaaaaaaaaaaaaaaaaaaaaaaaaaaaaaaaaaaaa

SEQ ID NO: 37 - DNA, 539 bp, Homo sapiens
cctttcgttgcctgatcgccgccatcatgggtcgcatgcatgctcccgggaagggcctgtcccagtcggctttaccctatcgacgcagcgtccccacttggttgaagttgacatctgacgacgtgaaggagcagatttacaaactggccaagaagggccttactccttcacagatcggtgtaatcctgagagattcacatggtgttgcacaagtacgttttgtgacaggcaataaaattttaagaattcttaagtctaagggacttgctcctgatcttcctgaagatctataccatttaattaagaaagcagttgctgttcgaaagcatcttgagaggaacagaaaggataaggatgctaaattccgtctgattctaatagagagccggattcaccgtttggctcgatattataagaccaagcgagtcctccctcccaattggaaatatgaatcatctacagcctctgccctggtcgcataaatttgtctgtgtactcaagcaataaaatgattgtttaactaaaaaaaaaaaaaaaaa

SEQ ID NO: 38 - DNA, 566 bp, Homo sapiens
ctctttccggtgtggagtctggagacgacgtgcagaaatggcacctcgaaaggggaaggaaaagaaggaagaacaggtcatcagcctcggacctcaggtggctgaaggagagaatgtatttggtgtctgccatatctttgcatccttcaatgacacttttgtccatgtcactgatctttctggcaaagaaaccatctgccgtgtgactggtgggatgaaggtaaaggcagaccgagatgaatcctcaccatatgctgctatgttggctgcccaggatgtggcccagaggtgcaaggagctgggtatcaccgccctacacatcaaactccgggccacaggaggaaataggaccaagacccctggacctggggcccagtcggccctcagagcccttgcccgctcgggtatgaagatcgggcggattgaggatgtcacccccatcccctctgacagcactcgcaggaaggggggtcgccgtggtcgccgtctgtgaacaagattcctcaaaatattttctgttaataaattgccttcatgtaaactgttaaaaaaaaaaaaaaaaaaaa

SEQ ID NO: 39 - DNA, 539 bp, Homo sapiens
ggcaagatggcagaagtagagcagaagaagaagcggaccttccgcaagttcacctaccgcggcgtggacctcgaccagctgctggacatgtcctacgagcagctgatgcagctgtacagtgcgcgccagcggcggcggctgaaccggggcctgcggcggaagcagcactccctgctgaagcgcctgcgcaaggccaagaaggaggcgccgcccatggagaagccggaagtggtgaagacgcacctgcgggacatgatcatcctacccgagatggtgggcagcatggtgggcgtctacaacggcaagaccttcaaccaggtggagatcaagcccgagatgatcggccactacctgggcgagttctccatcacctacaagcccgtaaagcatggccggcccggcatcggggccacccactcctcccgcttcatccctctcaagtaatggctcagctaataaaggcgcacatgactccaaaaaaaaaaaaaaaaaaaaaaaaaaaaaaaaaaaaaaaaaaaaaaaaaaaaaaaaaaaaaaaa

SEQ ID NO: 40 - DNA, 1083 bp, Homo sapiens
gggggaagatggcggccctcaaggctctggtgtccggctgtgggcggcttctccgtgggctactagcgggcccggcagcgaccagctggtctcggcttccagctcgcgggttcagggaagtggtggagacccaagaagggaagacaactataattgaaggccgtatcacagcgactcccaaggagagtccaaatcctcctaacccctctggccagtgccccatctgccgttggaacctgaagcacaagtataactatgacgatgttctgctgcttagccagttcatccggcctcatggaggcatgctgccccgaaagatcacaggcctatgccaggaagaacaccgcaagatcgaggagtgtgtgaagatggcccaccgagcaggtctattaccaaatcacaggcctcggcttcctgaaggagttgttccgaagagcaaaccccaactcaaccggtacctgacgcgctgggctcctggctccgtcaagcccatctacaaaaaaggcccccgctggaacagggtgcgcatgcccgtggggtcaccccttctgagggacaatgtctgctactcaagaacaccttggaagctgtatcactgacagagagcagtgcttccagagttcctcctgcacctgtgctggggagtaggaggcccactcacaagcccttggccacaactatactcctgtcccaccccaccacgatggcctggtccctccaacatgcatggacaggggacagtgggactaacttcagtacccttggcctgcacagtagcaatgctgggagctagaggcaggcagggcagttgggtcccttgccagctgctatggggcttaggccatgctcagtgctggggacaggagttttgcccaacgcagtgtcataaactgggttcatgggcttacccattgggtgtgcgctcactgcttgggaagtgcagggggtcctgggcacattgccagctgggtgctgagcattgagtcactgatctcttgtgatggggccaatgagtcaattgaattcatgggccaaacaggtcccatcctctgcaaaaaaaaaaaaaaaaaaaaaaaaaaaaaa

SEQ ID NO: 41 - DNA, 517 bp, Homo sapiens
gaggatttttggtccgcacgctcctgctcctgactcaccgctgttcgctctcgccgaggaacaagtcggtcaggaagcccgcgcgcaacagccatggcttttaaggataccggaaaaacacccgtggagccggaggtggcaattcaccgaattcgaatcaccctaacaagccgcaacgtaaaatccttggaaaaggtgtgtgctgacttgataagaggcgcaaaagaaaagaatctcaaagtgaaaggaccagttcgaatgcctaccaagactttgagaatcactacaagaaaaactccttgtggtgaaggttctaagacgtgggatcgtttccagatgagaattcacaagcgactcattgacttgcacagtccttctgagattgttaagcagattacttccatcagtattgagccaggagttgaggtggaagtcaccattgcagatgcttaagtcaactattttaataaattgatgaccagttgttaaaaaaaaaaaaaaaaaaaaaaaaaaaaaa

SEQ ID NO: 42 - DNA, 994 bp, Homo sapiens
gcttctctctttcgctcaggcccgtggcgccgacaggatgggcaagtgtcgtggacttcgtactgctaggaagctccgtagtcaccgacgagaccagaagtggcatgataaacagtataagaaagctcatttgggcacagccctaaaggccaacccttttggaggtgcttctcatgcaaaaggaatcgtgctggaaaaagtaggagttgaagccaaacagccaaattctgccattaggaagtgtgtaagggtccagctgatcaagaatggcaagaaaatcacagcctttgtacccaatgacggttgcttgaactttattgaggaaaatgatgaagttctggttgctggatttggtcgcaaaggtcatgctgttggtgatattcctggagtccgctttaaggttgtcaaagtagccaatgtttctcttttggccctatacaaaggcaagaaggaaagaccaagatcataaatattaatggtgaaaacactgtagtaataaattttcatatgccaaaaaatgtttgtatcttactgtcccctgttctcaccatgaagatcatgttcattaccaccaccacccccccttattttttttatcctaaaccagcaaacgcaggacctgtaccaattttaggagacaataagacagggttgtttcaggattctctagagttaataacatttgtaacctggcacagtttccctcatcctgtggaataagaaaatgagatagatctggaataaatgtgcagtattgtagtattactttaagaactttaagggaacttcaaaaactcactgaaattctagtgagatactttcttttttattcttggtattttccatatcgggtgcaacacttcagttaccaaatttcattgcacatagattatcttaggtacccttggaaatgcacattcttgtatccatcttacaggggcccaagatgataaatagtaaactcaaaaaaaaaaaaaaaaaaaaaaaaaaaaaa

SEQ ID NO: 43 - DNA, 481 bp, Homo sapiens
cctttccggcggtgacgacctacgcacacgagaacatgcctctcgcaaaggatctccttcatccctctccagaagaggagaagaggaaacacaagaagaaacgcctggtgcagagccccaattcctacttcatggatgtgaaatgcccaggatgctataaaatcaccacggtctttagccatgcacaaacggtagttttgtgtgttggctgctccactgtcctctgccagcctacaggaggaaaagcaaggcttacagaaggatgttccttcaggaggaagcagcactaaaagcactctgagtcaagatgagtgggaaaccatctcaataaacacattttggataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - DNA, 500 bp, Homo sapiens
tccgccagaccgccgccgcgccgccatcatggacaccagccgtgtgcagcctatcaagctggccagggtcaccaaggtcctgggcaggaccggttctcagggacagtgcacgcaggtgcgcgtggaattcatggacgacacgagccgatccatcatccgcaatgtaaaaggccccgtgcgcgagggcgacgtgctcacccttttggagtcagagcgagaagcccggaggttgcgctgagcttggctgctcgctgggtcttggatgtcgggttcgaccacttggccgatgggaatggtctgtcacaatctgctccttttttttgtccgccacacgtaactgagatgctcctttaaataaagcgtttgtgtttcaagttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - DNA, 1305 bp, Homo sapiens
cggacgcgtgggttgatggcgtgatgtctcacagaaagttctccgctcccagacatgggtccctcggcttcctgcctcggaagcgcagcagcaggcatcgtgggaaggtgaagagcttccctaaggatgacccatccaagccggtccacctcacagccttcctgggatacaaggctggcatgactcacatcgtgcgggaagtcgacaggccgggatccaaggtgaacaagaaggaggtggtggaggctgtgaccattgtagagacaccacccatggtggttgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgctcgtgtagccttctctgtggcacgcgctgggcagaaaggctaccatcaccgcactgagatcaacaagaagatttataagattggccagggctaccttatcaaggacggcaagctgatcaagaacaatgcctccactgactatgacctatctgacaagagcatcaaccctctgggtggctttgtccactatggtgaagtgaccaatgactttgtcatgctgaaaggctgtgtggtgggaaccaagaagcgggtgctcaccctccgcaagtccttgctggtgcagacgaagcggcgggctctggagaagattgaccttaagttcattgacaccacctccaagtttggccatggccgcttccagaccatggaggagaagaaagcattcatgggaccactgaagaaagaccgaattgcaaaggaagaaggagcttaatgccaggaacagattttgcagttggtggggtctcaataaaagttattttccactgaaaaaaaaaaaaaaa

SEQ ID NO: 46 - DNA, 831 bp, Homo sapiens
ggaaccatggagggtgtagaagagaagaagaaggaggttcctgctgtgccagaaacccttaagaaaaagcgaaggaatttcgcagagctgaagatcaagcgcctgagaaagaagtttgcccaaaagatgcttcgaaaggcaaggaggaagcttatctatgaaaaagcaaagcactatcacaaggaatataggcagatgtacagaactgaaattcgaatggcgaggatggcaagaaaagctggcaacttctatgtacctgcagaacccaaattggcgtttgtcatcagaatcagaggtatcaatggagtgagcccaaaggttcgaaaggtgttgcagcttcttcgccttcgtcaaatcttcaatggaacctttgtgaagctcaacaaggcttcgattaacatgctgaggattgtagagccatatattgcatgggggtaccccaatctgaagtcagtaaatgaactaatctacaagcgtggttatggcaaaatcaataagaagcgaattgctttgacagataacgctttgattgctcgatctcttggtaaatacggcatcatctgcatggaggatttgattcatgagatctatactgttggaaaacgcttcaaagaggcaaataacttcctgtggcccttcaaattgtcttctccacgaggtggaatgaagaaaaagaccacccattttgtagaaggtggagatgctggcaacagggaggaccagatcaacaggcttattagaagaatgaactaaggtgtctaccatgattatttttctaagctggttggttaataaacagtacctgctctcaaattgaaaaaaaaaaaaaaa

SEQ ID NO: 47 - DNA, 892 bp, Homo sapiens
gatgccgaaaggaaagaaggccaagggaaagaaggtggctccggccccagctgtcgtgaagaagcaggaggctaagaaagtggtgaatcccctgtttgagaaaaggcctaagaattttggcattggacaggacatccagcccaaaagagacctcacccgctttgtgaaatggccccgctatatcaggttgcagcggcagagagccatcctctataagcggctgaaagtgcctcctgcgattaaccagttcacccaggccctggaccgccaaacagctactcagctgcttaagctggcccacaagtacagaccagagacaaagcaagagaagaagcagagactgttggcccgggccgagaagaaggctgctggcaaaggggacgtcccaacgaagagaccacctgtccttcgagcaggagttaacaccgtcaccaccttggtggagaacaagaaagctcagctggtggtgattgcacacgacgtggatcccatcgagctggttgtcttcttgcctgccctgtgtcgtaaaatgggggtcccttactgcattatcaagggaaaggcaagactgggacgtctagtccacaggaagacctgcaccactgtcgccttcacacaggtgaactcggaagacaaaggcgctttggctaagctggtggaagctatcaggaccaattacaatgacagatacgatgagatccgccgtcactggggtggcaatgtcctgggtcctaagtctgtggctcgtatcgccaagctcgaaaaggcaaaggctaaagaacttgccactaaactgggttaaatgtacactgttgagttttctgtacataaaaataattgaaataatacaaattttccttcaaaaaaaaaaaaaaaaaaaaaaaaaaaaaaa

SEQ ID NO: 48 - DNA, 744 bp, Homo sapiens
tgaagatcctggtgtcgccatgggccgccgccccgcccgttgttaccggtattgtaagaacaagccgtacccaaagtctcgcttctgccgaggtgtccctgatgccaagattcgcatttttgacctggggcggaaaaaggcaaaagtggatgagtttccgctttgtggccacatggtgtcagatgaatatgagcagctgtcctctgaagccctggaggctgcccgaatttgtgccaataagtacatggtaaaaagttgtggcaaagatggcttccatatccgggtgcggctccaccccttccacgtcatccgcatcaacaagatgttgtcctgtgctggggctgacaggctccaaacaggcatgcgaggtgcctttggaaagccccagggcactgtggccagggttcacattggccaagttatcatgtccatccgcaccaagctgcagaacaaggagcatgtgattgaggccctgcgcagggccaagttcaagtttcctggccgccagaagatccacatctcaaagaagtggggcttcaccaagttcaatgctgatgaatttgaagacatggtggctgaaaagcggctcatcccagatggctgtggggtcaagtacatccccaatcgtggccctctggacaagtggcgggccctgcactcatgagggcttccaatgtgctgcccccctcttaatactcaccaataaattctacttcctgtccaaaaaaaaaaaaaaaaaaaaaa

SEQ ID NO: 49 - DNA, 1296 bp, Homo sapiens
ctgggtcctggcctttgggcatcatccagcgccatcggcctggcgcttcagccaacgcgggagtggatgggccccttcttcttcgcagacagcgttcggccgctgcccgggctctaggcgcggccggacggcccagtctggagggttcggggcggaggcccgggggggtgcgcgcgcccggggtccggcctctcactcgctcccctctcgtccgcagccgcagggccgtaggcagccatggcgcccagccggaatggcatggtcttgaagccccacttccacaaggactggcagcggcgcgtggccacgtggttcaaccagccggcccgtaagatccgcagacgtaaggcccggcaagccaaggcgcgccgcatcgccccgcgccccgcgtcgggtcccatccggcccatcgtgcgctgccccacggttcggtaccacacgaaggtgcgcgccggccgcggcttcagcctggaggagctcagggtggccggcattcacaagaaggtggcccggaccatcggcatttctgtggatccgaggaggcggaacaagtccacggagtccctgcaggccaacgtgcagcggctgaaggagtaccgctccaaactcatcctcttccccaggaagccctcggcccccaagaagggagacagttctgctgaagaactgaaactggccacccagctgaccggaccggtcatgcccgtccggaacgtctataagaaggagaaagctcgagtcatcactgaggaagagaagaatttcaaagccttcgctagtctccgtatggcccgtgccaacgcccggctcttcggcatacgggcaaaaagagccaaggaagccgcagaacaggatgttgaaaagaaaaaataaagccctcctggggacttggaatcagtcggcagtcatgctgggtctccacgtggtgtgtttcgtgggaacaactgggcctgggatggggcttcactgctgtgacttcctcctgccaggggatttggggctttcttgaaagacagtccaagccctggataatgctttactttctgtgttgaagcactgttggttgtttggttagtgactgatgtaaaacggttttcttgtggggaggttacagaggctgacttcagagtggacttgtgttttttctttttaaagaggcaaggttgggctggtgctcacagctgtaatcccagcactttgaggttggctgggagttcaagaccagcctggccaacatgtcagaactactaaaaataaagaaatcagccatgaaaaaaaaaaaaaaaaaaaaaaaaa

SEQ ID NO: 50 - DNA, 1126 bp, Homo sapiens
ccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 51 - DNA, 565 bp, Homo sapiens
atccagtccccttccttcggtgtttgagaccacttcatctggaccgagctaaagtctaggaagaaataaagtttcaaacccagtagagttacctcaaagatacacttgagacccttttcagaagatggcaccgaaagtgaagaaggaagctcctggcccgcctaaagctgaagccaaagcaaaggctttaaaggccaagaaggtagtgttgaaaggtgtccacggccacaaaaaaaagaagatccgcatgtcacccaccttccagcggcccaagacactgagactctggaggccgcccagatatcctcggaagaccacccccaggagaaacaagcttgaccactatgctatcatcaagtttcctctgaccactgagtttgccatgaagaagataaaagacaacaacacccttgtgttcactgtggatgttaaagccaacaagcaccagatcaaacaggctgtgaagaagctctgtgacattgatggggccaaggtcaacaccctgatggagagatgaaggcatatgttccactggctcctgattatgatgctttggatgttgcca

SEQ ID NO: 52 - DNA, 538 bp, Homo sapiens
ctttttcgtctgggctgccaacatgccatccagactgaggaagacccggaaacttaggggccacgtgagccacggccacggccgcataggcaagcaccggaagcaccccggcggccgcggtaatgctggtggtctgcatcaccaccggatcaacttcgacaaataccacccaggctactttgggaaagttggtatgaagcattacaacttaaagaggaaccagagcttctgcccaactgtcaaccttgacaaattgtggactttggtcagtgaacagacacgggtgaatgctgctaaaaacaagactggggctgctcccatcattgatgtggtgcgatcgggctactacaaagttctgggaaagggaaagctcgcaaagcagcctgtcatcgtgaaggccaaattattcagcagaagagctgaggagaagattaagagtgttgggggggcctgtgtcctggtggcttgaagccacatggagggagtttcattaaatgctaactacttttaaaaaaaaaaaaaaaaaaaaaaaaaaaaa

SEQ ID NO: 53 - DNA, 515 bp, Homo sapiens
tcgttccccggccatcttagcggctgctgttggttgggggccgtcccgctcctaaggcaggaagatggtggccgcaaagaagacgaaaaagtcgctggagtcgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaaaccttttcacctacaaaatttcacctgcaaaccttaaacctgcaaaattttcctttaataaaatttgcttgttttaaaaaaaagaaaaaaaaaaaaaaaaaaa

SEQ ID NO: 54 - DNA, 746 bp, Homo sapiens
ctttccaacttggacgctgcagaatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaagtaagttctccatcccataaagccatttaaattcattagaaaaatgtccttacctcttaaaatgtgaattcatctgttaagctaggggtgacacacgtcattgtaccctttttaaattgttggtgtgggaagatgctaaagaatgcaaaactgatccatatctgggatgtaaaaaggttgtggaaaatagaatgcccagacccgtctacaaaaggtttttagagttgaaatatgaaatgtgatgtgggtatggaaattgactgttacttcctttacagatctacagacagtcaatgtggatgagaactaatcgctgatcgtcagatcaaataaagttataaaattgcaaaaaaaaaaaaaaaaaaaaaaaaaaaaaa

SEQ ID NO: 55 - DNA, 1787 bp, Homo sapiens
gacctcctgggatcgcatctggagagtgcctagtattctgccagcttcggaaagggagggaaagcaagcctggcagaggcacccattccattcccagcttgctccgtagctggcgattggaagacactctgcgacagtgttcagtccctgggcaggaaagcctccttccaggattcttcctcacctggggccgcttcttccccaaaaggcatcatggccgccctcagaccccttgtgaagcccaagatcgtcaaaaagagaaccaagaagttcatccggcaccagtcagaccgatatgtcaaaattaagcgtaactggcggaaacccagaggcattgacaacagggttcgtagaagattcaagggccagatcttgatgcccaacattggttatggaagcaacaaaaaaacaaagcacatgctgcccagtggcttccggaagttcctggtccacaacgtcaaggagctggaagtgctgctgatgtgcaacaaatcttactgtgccgagatcgctcacaatgtttcctccaagaaccgcaaagccatcgtggaaagagctgcccaactggccatcagagtcaccaaccccaatgccaggctgcgcagtgaagaaaatgagtaggcagctcatgtgcacgttttctgtttaaataaatgtaaaaactgccatctggcatcttccttccttgattttaagtcttcagcttcttggccaacttagtttgccacagagattgttcttttgcttaagcccctttggaatctcccatttggaggggatttgtaaaggacactcagtccttgaacaggggaatgtggcctcaagtgcacagactagccttagtcatctccagttgaggctgggtatgaggggtacagacttggccctcacaccaggtaggttctgagacacttgaagaagcttgtggctcccaagccacaagtagtcattcttagccttgcttttgtaaagttaggtgacaagttattccatgtgatgcttgtgagaattgagaaaatatgcatggaaatatccagatgaatttcttacacagattcttacgggatgcctaaattgcatcctgtaacttctgtccaaaaagaacaggatgatgtacaaattgctcttccaggtaatccaccacggttaactggaaaagcactttcagtctcctataaccctcccaccagctgctgcttcaggtataatgttacagcagtttgccaaggcggggacctaactggtgacaattgagcctcttgactggtactcagaatttagtgacacgtggtcctgattttttttggagacggggtcttgctctcacccaggctgggagtgcagtggcacactgactacagccttgacctccccaggctcaggtgatcttcccacctcagccttccaagtagctgggactacagatgcacacctccaaacctgggtagtttttgaagtttttttgtagaggtggtctagccatgttgcctaggctcccgaactcctgagctcaagcaatcctgcttcagcctcccaaagtactgggattacaggcatcttctgtagtatataggtcatgagggatatgggatgtggtacttatgagacagaaatgcttacaggatgtttttctgtaaccatcctggtcaacttagcagaaatgctgcgctgggtataataaagcttttctacttctagtctagacaggaatcttacagattgtctcctgttcaaaacctagtcataaatatttataatgcaaactggtcaaaaaaaaaaaaaaaaaa

SEQ ID NO: 56 - DNA, 1274 bp, Homo sapiens
ctaggtcgcggcgacatggccaaacgtaccaagaaagtcgggatcgtcggtaaatacgggacccgctatggggcctccctccggaaaatggtgaagaaaattgaaatcagccagcacgccaagtacacttgctctttctgtggcaaaactaagatgaagagacgagctgtggggatctggcactgtggttcctgcatgaagacagtggctggcggtgcctggacgtacaataccacttccgctgtcacggtaaagtccgccatcagaagactgaaggagttgaaagaccagtagacgctcctctactctttgagacatcactggcctataataaatgggttaatttatgtaacaaaattgccttggcttgttaactttattagacattctgatgtttgcattgtgtaaatactgttgtattggaaaagcatgccaagatggattattgtaattcagtgtcttttttagtagtcaaatggtaaaatgcagcataagaatataagtcttccaagttagatatgagtgttagctttttataagtctgctcctgccagtttgactttgagatacattggagccaactgtaaactttagtttttaaattacagttagtttttttgtttgtttttgaggcggagtctctgttacccaggctggagtgcagtataccagtcttggcccacttcaacctccacttcttgggttcaagcgattctcctgcctcagcctcctgagtagctggggttgcaggcacgcgccaccatacctggctgatttttgtattttgagtagagatggagttttcaccacattggccaggctgttcttgaactgacctcaagcgatccacctgccttggccttccggagtgctgggattgcaggtgtgagccaccacgcccagccttgcatttaatatttttataatgtgtctaggctgggtgcggtgactcacgcctgaagtcccggcactttgggtggctgaggcgggtggattacttgaggccaggagattgagaccagtgtggccaacatagcaaaaacccgtctcgacgaaaaatacaaagaatagcttggtatggtggcgcgtgcctgtagtcccagctactttggaggctcaggcacaagagtcgcttgaacctacgaggcggaggttgcagtgagccaggatcgtgccactgcactttatttagccaggacaacactctgtctccaaaaaaaagtttctgaaggtaaaagatatactaaaggatatacaaaaaaaaaaaaaaaaaa

SEQ ID NO: 57 - DNA, 349 bp, Homo sapiens
ctctagggtgatacgtgggtgagaaaggtcctggtccgcgccagagcccagcgcgcctcgtcgccatgcctcggaaaattgaggaaatcaaggacttcctgctcacagcccgacgaaaggatgccaaatctgtcaagatcaagaaaaataaggacaacgtgaagtttaaagttcgatgcagcagatacctttacaccctggtcatcactgacaaagagaaggcagagaaactgaagcagtccctgccccccggtttggcagtgaaggaactgaaatgaaccagacacactgattggaactgtattatattaaaatactaaaaatccaaaaaaaaaaaaaaaaaaaaaaa

SEQ ID NO: 58 - DNA, 419 bp, Homo sapiens
cctcctcttcctttctccgccatcgtggtgtgttcttgactccgctgctcgccatgtcttctcacaagactttcaggattaagcgattcctggccaagaaacaaaagcaaaatcgtcccattccccagtggattcggatgaaaactggaaataaaatcaggtacaactccaaaaggagacattggagaagaaccaagctgggtctataaggaattgcacatgagatggcacacatatttatgctgtctgaaggtcacgatcatgttaccatatcaagctgaaaatgtcaccactatctggagatttcgacgtgttttcctctctgaatctgttatgaacacgttggttggctggattcagtaataaatatgtaaggcctttctttttagaaaaaaaaaaaaaaaaaaaaaaaaaaaaaa

SEQ ID NO: 59 - DNA, 607 bp, Homo sapiens
cttgctgcgacgcagcggtcggaagcggagcaaggtcgaggccgggttggcgccggagccggggccgcttggagctcgtgtggggtctccggtccagggcgcggcatgggcgtcctggccgcagcggcgcgctgcctggtccggggtgcggaccgaatgagcaagtggacgagcaagcggggcccgcgcagcttcaggggccgcaagggccggggcgccaagggcatcggcttcctcacctcgggctggaggttcgtgcagatcaaggagatggtcccggagttcgtcgtcccggatctgaccggcttcaagctcaagccctacgtgagctacctcgcccctgagagcgaggagacgcccctgacggccgcgcagctcttcagcgaagccgtggcgcctgccatcgaaaaggacttcaaggacggtaccttcgaccctgacaacctggaaaagtacggcttcgagcccacacaggagggaaagctcttccagctctaccccaggaacttcctgcgctagctgggcgggggaggggcggcctgccctcatctcatttctattaaacgcctttgccagctaaaaaaaaaaaaaaaaaaaaaaaaaaaaa

SEQ ID NO: 60 - RNA, 1871 nt, Homo sapiens
uaccugguugauccugccaguagcauaugcuugucucaaagauuaagccaugcaugucuaaguacgcacggccgguacagugaaacugcgaauggcucauuaaaucaguuaugguuccuuuggucgcucgcuccucuccuacuuggauaacugugguaauucuagagcuaauacaugccgacgggcgcugacccccuucgcgggggggaugcgugcauuuaucagaucaaaaccaacccggucagccccucuccggccccggccggggggcgggcgccggcggcuuuggugacucuagauaaccucgggccgaucgcacgccccccguggcggcgacgacccauucgaacgucugcccuaucaacuuucgaugguagucgccgugccuaccauggugaccacgggugacggggaaucaggguucgauuccggagagggagccugagaaacggcuaccacauccaaggaaggcagcaggcgcgcaaauuacccacucccgacccggggagguagugacgaaaaauaacaauacaggacucuuucgaggcccuguaauuggaaugaguccacuuuaaauccuuuaacgaggauccauuggagggcaagucuggugccagcagccgcgguaauuccagcuccaauagcguauauuaaaguugcugcaguuaaaaagcucguaguuggaucuugggagcgggcgggcgguccgccgcgaggcgagccaccgcccguccccgccccuugccucucggcgcccccucgaugcucuuagcugagugucccgcggggcccgaagcguuuacuuugaaaaaauuagaguguucaaagcaggcccgagccgccuggauaccgcagcuaggaauaauggaauaggaccgcgguucuauuuuguugguuuucggaacugaggccaugauuaagagggacggccgggggcauucguauugcgccgcuagaggugaaauucuuggaccggcgcaagacggaccagagcgaaagcauuugccaagaauguuuucauuaaucaagaacgaaagucggagguucgaagacgaucagauaccgucguaguuccgaccauaaacgaugccgaccggcgaugcggcggcguuauucccaugacccgccgggcagcuuccgggaaaccaaagucuuuggguuccggggggaguaugguugcaaagcugaaacuuaaaggaauugacggaagggcaccaccaggaguggagccugcggcuuaauuugacucaacacgggaaaccucacccggcccggacacggacaggauugacagauugauagcucuuucucgauuccgugggugguggugcauggccguucuuaguugguggagcgauuugucugguuaauuccgauaacgaacgagacucuggcaugcuaacuaguuacgcgacccccgagcggucggcgucccccaacuucuuagagggacaaguggcguucagccacccgagauugagcaauaacaggucugugaugcccuuagauguccggggcugcacgcgcgcuacacugacuggcucagcgugugccuacccuacgccggcaggcgcggguaacccguugaaccccauucgugauggggaucggggauugcaauuauuccccaugaacgagggaauucccgaguaagugcgggucauaagcuugcguugauuaagucccugcccuuuguacacaccgcccgucgcuacuaccgauuggaugguuuagugaggcccucggaucggccccgccggggucggcccacggcccuggcggagcgcugagaagacggucgaacuugacuaucuagaggaaguaaaagucguaacaagguuuccguaggugaaccugcggaaggaucauua

SEQ ID NO: 61 - RNA, 5035 nt, Homo sapiens
cgcgaccucagaucagacguggcgacccgcugaauuuaagcauauuagucagcggaggaaaagaaacuaaccaggauucccucaguaacggcgagugaacagggaagagcccagcgccgaauccccgccccgcggggcgcgggacauguggcguacggaagacccgcuccccggcgccgcucguggggggcccaaguccuucugaucgaggcccagcccguggacggugugaggccgguagcggccggcgcgcgcccgggucuucccggagucggguugcuugggaaugcagcccaaagcgggugguaaacuccaucuaaggcuaaauaccggcacgagaccgauagucaacaaguaccguaagggaaaguugaaaagaacuuugaagagagaguucaagagggcgugaaaccguuaagagguaaacgggugggguccgcgcaguccgcccggaggauucaacccggcggcggguccggccgugucggcggcccggcggaucuuucccgccccccguuccucccgaccccuccacccgcccucccuucccccgccgccccuccuccuccuccccggagggggcgggcuccggcgggugcgggggugggcgggcggggccggggguggggucggcgggggaccgucccccgaccggcgaccggccgccgccgggcgcauuuccaccgcggcggugcgccgcgaccggcuccgggacggcugggaaggcccggcggggaagguggcucggggggccccguccguccguccguccuccuccucccccgucuccgccccccggccccgcguccucccucgggagggcgcgcgggucggggcggcggcggcggcggcgguggcggcggcggcgggggcggcgggaccgaaaccccccccgaguguuacagcccccccggcagcagcacucgccgaaucccggggccgagggagcgagacccgucgccgcgcucuccccccucccggcgcccacccccgcggggaaucccccgcgaggggggucucccccgcgggggcgcgccggcgucuccucgugggggggccgggccaccccucccacggcgcgaccgcucucccaccccuccuccccgcgcccccgccccggcgacggggggggugccgcgcgcgggucggggggcggggcggacuguccccagugcgccccgggcgggucgcgccgucgggcccgggggagguucucucggggccacgcgcgcgucccccgaagagggggacggcggagcgagcgcacggggucggcggcgacgucggcuacccacccgacccgucuugaaacacggaccaaggagucuaacacgugcgcgagucgggggcucgcacgaaagccgccguggcgcaaugaaggugaaggccggcgcgcucgccggccgaggugggaucccgaggccucuccaguccgccgagggcgcaccaccggcccgucucgcccgccgcgccggggagguggagcacgagcgcacguguuaggacccgaaagauggugaacuaugccugggcagggcgaagccagaggaaacucugguggagguccguagcgguccugacgugcaaaucggucguccgaccuggguauaggggcgaaagacuaaucgaaccaucuaguagcugguucccuccgaaguuucccucaggauagcuggcgcucucgcagacccgacgcacccccgccacgcaguuuuauccgguaaagcgaaugauuagaggucuuggggccgaaacgaucucaaccuauucucaaacuuuaaauggguaagaagcccggcucgcuggcguggagccgggcguggaaugcgagugccuagugggccacuuuugguaagcagaacuggcgcugcgggaugaaccgaacgccggguuaaggcgcccgaugccgacgcucaucagaccccagaaaagguguugguugauauagacagcaggacgguggccauggaagucggaauccgcuaaggaguguguaacaacucaccugccgaaucaacuagcccugaaaauggauggcgcuggagcgucgggcccauacccggccgucgccggcagucgagaguggacgggagcggcgggggcggcgcgcgcgcgcgcgcguguggugugcgucggagggcggcggcggcggcggcggcgggggugugggguccuucccccgcccccccccccacgccuccuccccuccucccgcccacgccccgcuccccgcccccggagccccgcggacgcuacgccgcgacgaguaggagggccgcugcggugagccuugaagccuagggcgcgggcccggguggagccgccgcaggugcagaucuuggugguaguagcaaauauucaaacgagaacuuugaaggccgaaguggagaaggguuccaugugaacagcaguugaacaugggucagucgguccugagagaugggcgagcgccguuccgaagggacgggcgauggccuccguugcccucggccgaucgaaagggagucggguucagauccccgaauccggaguggcggagaugggcgccgcgaggcguccagugcgguaacgcgaccgaucccggagaagccggcgggagccccggggagaguucucuuuucuuugugaagggcagggcgcccuggaauggguucgccccgagagaggggcccgugccuuggaaagcgucgcgguuccggcggcguccggugagcucucgcuggcccuugaaaauccgggggagaggguguaaaucucgcgccgggccguacccauauccgcagcaggucuccaaggugaacagccucuggcauguuggaacaauguagguaagggaagucggcaagccggauccguaacuucgggauaaggauuggcucuaagggcugggucggucgggcuggggcgcgaagcggggcugggcgcgcgccgcggcuggacgaggcgcgcgccccccccacgcccggggcaccccccucgcggcccucccccgccccacccgcgcgcgccgcucgcucccuccccaccccgcgcccucucucucucucucucccccgcuccccguccuccccccuccccgggggagcgccgcgugggggcgcggcggggggagaagggucggggcggcaggggccgcgcggcggccgccggggcggccggcgggggcagguccccgcgaggggggccccggggacccggggggccggcggcggcgcggacucuggacgcgagccgggcccuucccguggaucgccccagcugcggcgggcgucgcggccgcccccggggagcccggcggcggcgcggcgcgccccccacccccaccccacgucucggucgcgcgcgcguccgcugggggcgggagcggucgggcggcggcggucggcgggcggcggggcggggcgguucguccccccgcccuacccccccggccccguccgccccccguuccccccuccuccucggcgcgcggcggcggcggcggcaggcggcggaggggccgcgggccggucccccccgccggguccgcccccggggccgcgguuccgcgcgcgccucgccucggccggcgccuagcagccgacuuagaacuggugcggaccaggggaauccgacuguuuaauuaaaacaaagcaucgcgaaggcccgcggcggguguugacgcgaugugauuucugcccagugcucugaaugucaaagugaagaaauucaaugaagcgcggguaaacggcgggaguaacuaugacucucuuaagguagccaaaugccucgucaucuaauuagugacgcgcaugaauggaugaacgagauucccacugucccuaccuacuauccagcgaaaccacagccaagggaacgggcuuggcggaaucagcggggaaagaagacccuguugagcuugacucuagucuggcacggugaagagacaugagagguguagaauaagugggaggcccccggcgcccccccgguguccccgcgaggggcccggggcgggguccgcggcccugcgggccgccggugaaauaccacuacucugaucguuuuuucacugacccggugaggcgggggggcgagcccgaggggcucucgcuucuggcgccaagcgcccgcccggccgggcgcgacccgcuccggggacagugccagguggggaguuugacuggggcgguacaccugucaaacgguaacgcagguguccuaaggcgagcucagggaggacagaaaccucccguggagcagaagggcaaaagcucgcuugaucuugauuuucaguacgaauacagaccgugaaagcggggccucacgauccuucugaccuuuuggguuuuaagcaggaggugucagaaaaguuaccacagggauaacuggcuuguggcggccaagcguucauagcgacgucgcuuuuugauccuucgaugucggcucuuccuaucauugugaagcagaauucgccaagcguuggauuguucacccacuaauagggaacgugagcuggguuuagaccgucgugagacagguuaguuuuacccuacugaugauguguuguugccaugguaauccugcucaguacgagaggaaccgcagguucagacauuugguguaugugcuuggcugaggagccaauggggcgaagcuaccaucugugggauuaugacugaacgccucuaagucagaaucccgcccaggcgaacgauacggcagcgccgcggagccucgguuggccucggauagccggucccccgccuguccccgccggcgggccgccccccccuccacgcgccccgccgcgggagggcgcgugccccgccgcgcgccgggaccgggguccggugcggagugcccuucguccugggaaacggggcgcggccggaaaggcggccgcccccucgcccgucacgcaccgcacguucguggggaaccuggcgcuaaaccauucguagacgaccugcuucugggucgggguuucguacguagcagagcagcucccucgcugcgaucuauugaaagucagcccucgacacaaggguuuguc

SEQ ID NO: 62 - RNA, 140 nt, Homo sapiens
cgacucuuagcgguggaucacucggcucgugcgucgaugaagaacgcagcuagcugcgagaauuaaugugaauugcaggacacauugaucaucgacacuucgaacgcacuugcggccccggguuccucccggggcuacgc

SEQ ID NO: 63 - DNA, 21 bp, Artificial Sequence
  Description of Artificial Sequence  Synthetic Primer
gccgcccactcagactttatt

SEQ ID NO: 64 - DNA, 16 bp, Artificial Sequence
  Description of Artificial Sequence  Synthetic Primer
aaagaccacgggggta

SEQ ID NO: 65 - DNA, 12 bp, Artificial Sequence
  Description of Artificial Sequence  Synthetic Primer
ccactcagactt

SEQ ID NO: 66 - DNA, 11 bp, Artificial Sequence
  Description of Artificial Sequence  Synthetic Primer
aaagaccacgg

SEQ ID NO: 67 - DNA, 12 bp, Artificial Sequence
  Description of Artificial Sequence  Synthetic Primer
ccactcagactt

SEQ ID NO: 68 - DNA, 11 bp, Artificial Sequence
  Description of Artificial Sequence  Synthetic Primer
aaagaccacgg

SEQ ID NO: 69 - DNA, 16 bp, Artificial Sequence
  Description of Artificial Sequence  Synthetic Primer
gcaatgaaaataaatg

SEQ ID NO: 70 - DNA, 22 bp, Artificial Sequence
  Description of Artificial Sequence  Synthetic Primer
tttattaggcagaatccagatg

SEQ ID NO: 71 - DNA, 15 bp, Artificial Sequence
  Description of Artificial Sequence  Synthetic Primer
tttattaggcagaat

SEQ ID NO: 72 - DNA, 14 bp, Artificial Sequence
  Description of Artificial Sequence  Synthetic Primer
aatgaaaataaatg

SEQ ID NO: 73 - DNA, 15 bp, Artificial Sequence
  Description of Artificial Sequence  Synthetic Primer
tttattaggcagaat

SEQ ID NO: 74 - DNA, 12 bp, Artificial Sequence
  Description of Artificial Sequence  Synthetic Primer
ttaccttatcct

SEQ ID NO: 75 - DNA, 13 bp, Artificial Sequence
  Description of Artificial Sequence  Synthetic Primer
cgccaagataaaa

SEQ ID NO: 76 - DNA, 13 bp, Artificial Sequence
  Description of Artificial Sequence  Synthetic Primer
catccacttggac

SEQ ID NO: 77 - DNA, 13 bp, Artificial Sequence
  Description of Artificial Sequence  Synthetic Primer
ccttcctagtaat

SEQ ID NO: 78 - DNA, 13 bp, Artificial Sequence
  Description of Artificial Sequence  Synthetic Primer
gataagagtttga

SEQ ID NO: 79 - DNA, 13 bp, Artificial Sequence
  Description of Artificial Sequence  Synthetic Primer
atttacccattct

SEQ ID NO: 80 - DNA, 13 bp, Artificial Sequence
  Description of Artificial Sequence  Synthetic Primer
taggctgacaaat

SEQ ID NO: 81 - DNA, 13 bp, Artificial Sequence
  Description of Artificial Sequence  Synthetic Primer
aattttgtttcgt

SEQ ID NO: 82 - DNA, 13 bp, Artificial Sequence
  Description of Artificial Sequence  Synthetic Primer
tcagtcgggagct

SEQ ID NO: 83 - DNA, 13 bp, Artificial Sequence
  Description of Artificial Sequence  Synthetic Primer
tgttcccaaacag

SEQ ID NO: 84 - DNA, 12 bp, Artificial Sequence
  Description of Artificial Sequence  Synthetic Primer
ccccgatgcgga

SEQ ID NO: 85 - DNA, 13 bp, Artificial Sequence
  Description of Artificial Sequence  Synthetic Primer
gactcgcagcgaa

(19) United States
(12) Patent Application Publication
(10) Pub. No.: US 2009/0264635 A1
(43) Pub. Date: Oct. 22, 2009

(54) METHODS AND COMPOSITIONS FOR DEPLETING ABUNDANT RNA TRANSCRIPTS

(75) Inventors: Leopoldo Mendoza, San Diego, CA (US); Sharmili Moturi, JR., Austin, TX (US); Robert Setterquist, Austin, TX (US); John Penn Whitley, Austin, TX (US)

Correspondence Address: LIFE TECHNOLOGIES CORPORATION;C/O INTELLEVATE, P.O. BOX 52050, MINNEAPOLIS, MN, 55402, US

(73) Assignee: APPLERA CORPORATION, Foster City, CA (US)

(21) Appl. No.: 11/909,656

(22) PCT Filed: Mar. 27, 2006
(86) PCT No.: PCT/US06/11185

§ 371 Date: Jan. 16, 2008

Related U.S. Application Data
(60) Provisional application No. 60/665,453, filed on Mar. 25, 2005.

(51) Int. Cl. C07H 21/00; C07H 1/06; C07H 1/00

(52) U.S. Cl. 536/24.3; 536/25.4; 536/25.3

(57) ABSTRACT

The present invention concerns a system for isolating, depleting, and/or preventing the amplification of a targeted nucleic acid, such as mRNA or rRNA, from a sample comprising targeted and nontargeted nucleic acids.

The present application claims the benefit of U.S. Provisional Application Ser. No. 60/665,453 filed Mar. 25, 2005, the entire text of which is incorporated by reference.

### BACKGROUND OF THE INVENTION

1. Field of the Invention

The present invention relates generally to the fields of molecular biology and genetic analysis. More particularly, it concerns methods, compositions, and kits for isolating, depleting, or preventing the amplification of a targeted nucleic acid population in regard to other nucleic acid populations as a means for enriching those other nucleic acid population(s).

2. Description of Related Art

Genome wide expression profiling allows the simultaneous measurements of nearly all mRNA transcript levels present in a total RNA sample. Of the 25,000 to 30,000 unique genes present the human genome; any one tissue may be expressing tens of thousands of genes at various levels at any given time. Accurately determining differences between samples is the basis of understanding and associating genes and there products to a particular physiological state.

The amount of information that can be extracted from a sample is determined by many factors that are related to, the origin of the sample, the method used for global amplification, the limits of the instrumentation, and the methods used for analysis. Determining slight differences between samples (two-fold or less) requires that the entire process be highly reproducible. The ability to sample a large number of genes requires that the entire method produces signals from RNA transcripts reflective of the large range of concentrations (large dynamic range).

Current high density oligonucleotide microarrays, such as the Affymetrix GeneChip, have the content to interrogate nearly every human, rodent and other species genomes. The dynamic range is approximately 3 orders of magnitude and the technology can be used to profile expression patterns starting with a low number of cells.

All tissues contain RNA that can be utilized for global expression profiling. Some tissues are more difficult to study than others due to inefficient RNA extraction, low content of mRNA, limited size, or contain high concentrations of nucleases.

Blood is the most widely studied tissue in both clinical and research settings. Blood is easily obtained and contains biomolecules such as metabolites, enzymes, and antibodies that are very useful for monitoring a person's health. Increasingly, researchers and clinicians are using blood to monitor RNA expression profiles for medical research.

Blood is composed of plasma and hematic cells. There are several cell types that are classified in two groups, erythrocytes (red blood cells) and leukocytes (white blood cells). There are also platelets, which are not considered real cells. Red blood cells are the most numerous in blood. The ratio of red blood cells to white blood cells is approximately 700:1. Men average about 5 million red blood cells per microliter of blood and women have slightly less.

Red blood cells are responsible for the transport of oxygen and carbon dioxide. The red blood cells produce hemoglobin until it makes up about 90% of the dry weight of the cell. Two distinct globin chains (each with its individual heme molecule) combine to form hemoglobin. One of the chains is designated alpha. The second chain is called “non-alpha”. With the exception of the very first weeks of embryogenesis, one of the globin chains is always alpha. A number of variables influence the nature of the non-alpha chain in the hemoglobin molecule. The fetus has a distinct non-alpha chain called gamma. After birth, a different non-alpha globin chain, called beta, pairs with the alpha chain. The combination of two alpha chains and two non-alpha chains produces a complete hemoglobin molecule (a total of four chains per molecule).

The combination of two alpha chains and two gamma chains form “fetal” hemoglobin, termed “hemoglobin F”. With the exception of the first 10 to 12 weeks after conception, fetal hemoglobin is the primary hemoglobin in the developing fetus. The combination of two alpha chains and two beta chains form “adult” hemoglobin, also called “hemoglobin A”. Although hemoglobin A is called “adult”, it becomes the predominant hemoglobin within about 18 to 24 weeks of birth.

The pairing of one alpha chain and one non-alpha chain produces a hemoglobin dimer (two chains). The hemoglobin dimer does not efficiently deliver oxygen, however. Two dimers combine to form a hemoglobin tetramer, which is the functional form of hemoglobin. Complex biophysical characteristics of the hemoglobin tetramer permit the exquisite control of oxygen uptake in the lungs and release in the tissues that is necessary to sustain life.

The production of red blood cells occurs by a process called erythropoiesis whereby erythroid progenitor cells proliferate and differentiate into erythroid precursor cells. Normally, this process is highly dependent upon and regulated by a hormone produced by the kidneys called erythropoietin.

Immature red blood cells are called reticulocytes, and normally account for 0.8-2.0% of the circulating red blood cells. They are juvenile red cells produced by erythropoiesis which spend about 24 hours in the marrow before entering the peripheral circulation. They contain some nuclear material—remnants of RNA—which appears faintly blue—basophilic—in conventionally stained blood smears.

Reticulocytes persist for a few days in the circulation before forming the slightly smaller, mature red cell. Mature red blood cells do not contain a nucleus nor do they contain RNA. Reticulocytes contain significant amounts of RNA, mainly coding for needed globin protein subunits.

Total RNA isolated from whole blood (all cell types) will typically yield 1-5 ug RNA per milliliter of blood. Only a fraction of this RNA is mRNA (˜2%) and of this mRNA fraction up to 70% can be comprised of the globin mRNA transcripts derived from the reticulocytes. Because the white blood cells are actively transcribing RNA and constantly reacting to the changing physiology of the organism, these cells offer amble opportunity for diagnostic biomarkers, and studying the genetic responses to different disease and developmental states, or response to therapeutic treatments. However the low numbers of white blood cells compared to red blood cells and reticulocytes creates a disproportionate population of globin mRNA compared to the thousands of other mRNA in a whole blood RNA sample. Many low copy genes are effectively “diluted” by the abundant globin mRNA.

The presence of the two abundant globin transcripts can obscure global expression profiling methods. There is a need to eliminate these complications caused by globin or other abundant mRNA transcripts during microarray sample preparation.

Currently, a published method has been described for selectively removing globin mRNA prior to amplification. The method is based on RNase H cleavage of the 3′ ends of (α and β) globin transcripts hybridized to gene-specific primers (AFFYMETRIX TECHNICAL NOTES PUBLICATION). Total RNA treated in this manner is then purified from digestion products and reagents and the remaining ‘depleted’ RNA population is subsequently amplified using a conventional Eberwine amplification reaction.

A variant method has also been described (U.S. Pat. No. 6,391,592, assigned to Affymetrix). With this method non-extendable oligonucleotides that hybridize specifically to ribosomal transcripts and serve to block cDNA synthesis are used.

Nonetheless, such methods haves shortcomings. For example, RNase H treatment of RNA requires downstream purification and thus is not a homogeneous process. This limitation detracts from its utility (e.g. ease of use and cost) and also exposes the remaining sample RNA to potentially damaging nucleases (RNase H) and contaminating nucleases that may be present in the sample. Incubating RNA in a nuclease buffer at 37° C. prior to reverse transcription can lead to non-specific RNA degradation. The use of non-extendable rRNA specific oligonucleotides, although a homogeneous process, requires that the primers be blocked at their 3′-prime end using special chemical linkages or non-extendable nucleotides (e.g. inverted T or a dideoxy nucleotide terminators). These specialized 3′-blocked oligonucleotides serve to “block” reverse transcriptase from polymerizing through these hybridized, non-extendable blocking primers and thus impede upstream oligodT-T7 primed cDNA synthesis. This blocking method as described in has an absolute requirement that 3′-blocked primers be used, in effect, preventing them from serving as primers for initiating cDNA synthesis themselves. Thus, there remains a continued need for improvements in mRNA enrichment and/or the depletion of other RNA populations in general and for depletion and/or prevention of amplification of hemoglobin transcripts in particular.

### SUMMARY OF THE INVENTION

The present invention involves a system that allows for the depletion, isolation, separation, and/or prevention of amplification of a population of nucleic acid molecules. The system involves components that may be used to implement such methods and such components may also be included in kits of the invention.

In one aspect of the present invention, a population of RNA nucleic acids may be targeted such that the RNA amplification of such a population is selectively prevented. Such an RNA is termed a target or targeted RNA, or a target or targeted nucleic acid. In a typical embodiment, the RNA is a mRNA or rRNA. In some embodiments, the target RNA is targeted by a primer, which by definition is extendable and does not contain a phage polymerase promoter sequence. The primer comprises a targeting region that, in some embodiments, comprises between 6 to 30 nucleic acid residues complementary to the target RNA sequence. In a one embodiment, the primer targeting region is complementary to a sequence adjacent to the 3′ end of a mRNA. In another embodiment, the targeted nucleic acid is a rRNA sequence and the primer targeting region is complementary to a sequence that may be in the untranslated 5′ region, untranslated 3′ region, coding region, or may span such regions.

In some embodiments, the primer binds to a target mRNA in an RNA containing sample, and the sample conditions are adapted to provide for the extension of the primer by reverse transcription to form an DNA sequence complementary to that of the target RNA. A second primer comprising a poly(dT) sequence and a phage DNA polymerase promoter sequence is provided and the conditions adapted to support reverse transcription, wherein the first bound primer and the complementary DNA sequence prevents the full or efficient extension of the poly(dT) primer bound to the target mRNA, wherein such prevention is selective in regard to other non-targeted mRNA in the sample. In some embodiments, the conditions are adapted to partially degrade the RNA chains of RNA/DNA duplexes and second strand DNA sequences are synthesized to provide double stranded cDNAs, wherein the sense strands of those cDNAs derived from the target RNA are selectively devoid of a 3′-phage polymerase sequence in comparison to those sense strands of cDNAs derived from non-targeted mRNA. Thus, on purification or direct utilization of the cDNA and providing conditions adapted for in vitro transcription, the templates derived from targeted RNA are selectively prevented from synthesizing antisense RNA transcripts. This process is schematically summarized in FIG. 1. wherein the RNA-containing sample is a sample containing whole blood RNA and the target mRNA is a hemoglobin mRNA.

Another aspect of the present invention provides for the selective capture of a nucleic acid species or selected nucleic acid genus, either by direct or indirect means. Nucleic acids comprising a targeting regions are provided, wherein the targeting region comprises at least 5 contiguous nucleic acids complementary to the sequence of a target RNA. In some embodiments providing for direct capture, a capture nucleic acid comprises a targeting region, while in some embodiments providing for indirect capture, a bridging nucleic acid comprises a targeting region and a region complementary to part or whole of a capture nucleic acid.

Capture nucleic acids also includes a “non-reacting structure,” which refers to a moiety that does not chemically react with a nucleic acid. In some embodiments, a non-reacting structure is a super-paramagnetic bead or rod, which allows for the capture nucleic acid, a bridging nucleic acid (if used), and a target nucleic acid to be isolated from a sample with a magnetic field, such as a magnetic stand. In still further embodiments, the non-reacting structure is a bead or other structure that can be physically captured, such as by using a basket, filter, or by centrifugation. It is contemplated that a bead may include plastic, glass, teflon, silica, a magnet or be magnetizable, a metal such as a ferrous metal or gold, carbon, cellulose, latex, polystyrene, and other synthetic polymers, nylon, cellulose, agarose, nitrocellulose, polymethacrylate, polyvinylchloride, styrene-divinylbenzene, or any chemically-modified plastic or any other non-reacting structure. In still further embodiments the non-reacting structure is biotin or iminobiotin. Biotin or iminobiotin binds to avidin or streptavidin, which can be used to isolate the capture nucleic acid and any hybridizing molecules. In some embodiments, the streptavidin may be coated on the surface of a bead, which may be a super-paramagnetic bead.

FIG. 2 diagrammatically summarizes the components of the direct and indirect capture systems as exemplified by binding to a hemoglobin mRNA. FIG. 3 diagrammatically represents steps in a direct capture method utilizing a streptavidin/biotin system as exemplified by binding to a hemoglobin mRNA.

One aspect of the present invention is a method of depleting or preventing amplification of a RNA in a RNA-containing sample comprising: obtaining a RNA-containing sample; binding a nucleic acid to a RNA in the sample in a reaction mixture; and removing RNA bound to the nucleic acid from the reaction mixture and/or amplifying RNA not bound to the nucleic acid. In some embodiments, the binding of the nucleic acid to the RNA prevents RNA amplification of the RNA wherein the nucleic acid is a primer that does not comprise a polymerase promoter sequence, which may be a RNA polymerase promoter sequence, and is specific for the RNA. Embodiments also further comprising extending the primer to form a complementary DNA sequence. Further embodiments include addition of a primer comprising a polymerase promoter sequence, which may be an RNA polymerase promoter sequence, that anneals 3′ of the primer that does not comprise a RNA polymerase promoter sequence. In this context, in the phrase “anneals 3′ of the primer etc” the term “3′” refers to the 3′ end of the RNA to which the primers anneal, as shown in FIG. 1 in the context of mRNA. In some embodiments, the conditions in the reaction mixture are adapted to support reverse transcription and the extended bound primer that does not comprise a RNA polymerase promoter sequence prevents the extension of said primer comprising a RNA polymerase promoter sequence. In this context, the term “prevents” for the purposes of the present invention does not require complete prevention of the extension of the primer that comprises a RNA polymerase promoter sequence, but that full or efficient extension of the primer is prevented. In some embodiments, the RNA is a mRNA and the primer comprising a RNA polymerase promoter sequence is a poly(dT) primer comprising a phage RNA promoter polymerase promoter sequence, which may be a T3 polymerase promoter sequence, a T7 polymerase promoter sequence, or a SP2 polymerase promoter sequence. In some embodiments. The primer that does not comprise a RNA polymerase promoter sequence binds adjacent to the 3′ end of the mRNA and when extended prevents the extension of the poly(dT) primer comprising a phage polymerase promoter sequence. In some embodiments the mRNA is an abundant mRNA. In some embodiments the RNA is a rRNA. In typical embodiments, a plurality of primers that do not comprise a RNA polymerase primer bind to a target rRNA.

In some embodiments, the RNA is bound directly or indirectly to a capture nucleic acid, such as wherein the nucleic acid is a bridging nucleic acid adapted to bind to the RNA and to a capture nucleic acid. In some embodiments, the nucleic acid is a capture nucleic acid and binds directly to the RNA wherein the bound capture nucleic acid and RNA are removed from the reaction mixture prior to amplification. The removal may be facilitated by the capture nucleic acid being attached to a solid surface, wherein such attachment may be prior or after binding to the RNA. In some embodiments wherein the capture nucleic acid is attached to a solid surface after binding to the RNA, the capture nucleic acid is attached to the solid surface by covalent binding or via an biotin/streptavidin system. Embodiments include wherein the solid surface is a bead, a rod, or a plate. When the solid surface is a bead, it may comprise a super-paramagnetic material and a magnet may be used to remove the bead from the reaction mixture prior to amplification. In some embodiments the RNA is a mRNA, which may be an abundant mRNA. In other embodiments, the RNA is a rRNA, which may be an abundant RNA. In some embodiments, the direct or indirect binding of the capture nucleic acid to the RNA prevents the participation of the RNA or derived nucleic acids thereof in molecular biological procedures to which other RNA in the RNA sample are subjected to.

In embodiments wherein the mRNA is an abundant mRNA, the term “abundant mRNA” means for the purpose of the present invention, a mRNA present in a sample to an extent wherein the removal of that mRNA results in the increased fidelity in regard to the resulting RNA formed by RNA amplification of non-abundant mRNAs in the sample. In this context, “increased fidelity” means an increased yield of mRNA and/or a decreased 3′ bias of the amplified RNA. In some embodiments, an abundant mRNA is an mRNA that is at least 0.5% of the total mRNA in a sample. In some embodiments, the abundant mRNA is a hemoglobin chain mRNA. The term “hemoglobin chain” and “globin chain” are used interchangeably and refer to the chains subunits that comprise a globin protein. The hemoglobin chain mRNA may be a mammalian hemoglobin chain mRNA, which may be a primate or murine hemoglobin chain, which in turn may be human hemoglobin chain alpha 2 mRNA, or human hemoglobin beta chain mRNA. In some embodiments there are a plurality of primers that do not comprise a RNA polymerase promoter sequence or capture nucleic acids that bind to human hemoglobin chain alpha 1 mRNA, human hemoglobin chain alpha 2 mRNA, and human hemoglobin beta chain mRNA. In various embodiments, the abundant mRNA is actin beta mRNA, actin gamma 1 mRNA, calmodulin 2 (phosphorylase kinase, delta) mRNA, cofilin 1 (non-muscle) mRNA, eukaryotic translation elongation factor 1 alpha 1 mRNA, eukaryotic translation elongation factor 1 gamma mRNA, ferritin, heavy polypeptide pseudogene 1 mRNA, ferritin, light polypeptide mRNA, glyceraldehyde-3-phosphate dehydrogenase mRNA, GNAS complex locus mRNA, translationally-controlled 1 tumor protein mRNA, alpha tubulin mRNA, tumor protein mRNA, translationally-controlled 1 mRNA, ubiquitin B mRNA, or ubiquitin C mRNA, abundant mRNA is large ribosomal protein P0 mRNA, large ribosomal protein P1 mRNA, ribosomal protein S2, mRNA ribosomal protein S3A mRNA, X-linked ribosomal protein S4 mRNA, ribosomal protein S6 mRNA, ribosomal protein S10 mRNA, ribosomal protein S11 mRNA, ribosomal protein S13 mRNA, ribosomal protein S14 mRNA, ribosomal protein S15 mRNA, ribosomal protein S18 mRNA, ribosomal protein S20 mRNA, ribosomal protein S23 mRNA, ribosomal protein S27 (metallopanstimulin 1) mRNA, ribosomal protein S28 mRNA, ribosomal protein L3 mRNA, ribosomal protein L7 mRNA, ribosomal protein L7a mRNA, ribosomal protein L10 mRNA, ribosomal protein L13 mRNA, ribosomal protein L13a mRNA, ribosomal protein L23a mRNA, ribosomal protein L27a mRNA, ribosomal protein L30 mRNA, ribosomal protein L31 mRNA, ribosomal protein L32 mRNA, ribosomal protein L37a mRNA, ribosomal protein L38 mRNA, ribosomal protein L39 mRNA, or ribosomal protein L41 mRNA.

In embodiments wherein the RNA is an abundant RNA, the term “abundant RNA” means for the purpose of the present invention, a RNA present in a sample to an extent wherein the removal of that RNA results in the increased fidelity of the results of a subsequent use of the non-abundant RNAs in the sample, wherein such use involves, but is not limited to production of cDNA, amplification of DNA or RNA, and microarrays. In this context, “increased fidelity” includes removal of an RNA that would interfere with a desired result, increased yield, sensitivity, reproducibility of results, or the results are more representative of a RNA population. Abundant RNAs may be an rRNA, which may be s18S rRNA or 22S rRNA. In some embodiments, an abundant RNA is a RNA that is at least 50%, or 60%, or 70%, or 80% of the total RNA in a sample. In this regard, abundant RNAs are typically rRNA.

One aspect of the present invention is a method of selectively preventing the formation of a cDNA comprising a RNA polymerase promoter sequence from a RNA comprising: obtaining a RNA-containing sample; binding a primer that does not comprise a RNA polymerase promoter sequence to a RNA in the RNA-containing sample in a reaction mixture; and forming cDNAs from RNAs in said RNA-containing sample; wherein the binding of the primer that does not comprise a RNA polymerase promoter sequence selectively prevents the formation of a cDNA that does not contain a polymerase promoter sequence derived from said RNA.

Another aspect of the present invention is a method of preventing the reverse transcription of a RNA in a sample comprising: obtaining an RNA-containing sample; binding a nucleic acid to a RNA in the sample in a reaction mixture; reverse transcribing the RNA; wherein the binding of the nucleic acid to the RNA prevents reverse transcription of the RNA. Embodiments include wherein the RNA is bound directly or indirectly to a capture nucleic acid.

Aspects of the invention also encompass kits. One aspect provides for a kit in a suitable container, comprising a capture nucleic acid comprising a targeting region and a super-paramagnetic bead, wherein said targeting region comprising at least 5 nucleic acid bases complementary to the sequence of an RNA. In some embodiments the super-paramagnetic bead is coated by streptavidin and the capture nucleic acid comprises a biotin moiety. In some embodiments the RNA is a mRNA, which may be a hemoglobin mRNA. In some embodiments, the hemoglobin mRNA is SEQ ID NO: 1. The kit may further comprising a first capture nucleic acid comprising a targeting region comprising at least 5 nucleic acid bases complementary to SEQ ID NO: 1; a second capture nucleic acid comprising a targeting region comprising at least 5 nucleic acid bases complementary to SEQ ID NO: 2 and a third capture nucleic acid comprising a targeting region comprising at least 5 nucleic acid bases complementary to SEQ ID NO: 3. The kit may also further comprise a fourth capture nucleic acid, comprising a targeting region comprising at least 5 nucleic acid bases complementary to SEQ ID NO: 2; a fifth capture nucleic acid comprising a targeting region comprising at least 5 nucleic acid bases complementary to SEQ ID NO: 3; a sixth capture nucleic acid comprising a targeting region comprising at least 5 nucleic acid bases complementary to both SEQ ID NO: 1 and SEQ ID NO: 2; a seventh capture nucleic acid comprising a targeting region comprising at least 5 nucleic acid bases complementary to SEQ ID NO: 3; an eight capture nucleic acid comprising a targeting region comprising at least 5 nucleic acid bases complementary to SEQ ID NO: 3; a ninth capture nucleic acid comprising a targeting region comprising at least 5 nucleic acid bases complementary to SEQ ID NO: 3; and a tenth capture nucleic acid comprising a targeting region comprising at least 5 nucleic acid bases complementary to SEQ ID NO: 3. In some embodiments, the first capture nucleic acid comprises SEQ ID NO: 20; the second capture nucleic acid comprises SEQ ID NO: 19; the third capture nucleic acid comprises SEQ ID NO: 24; the fourth capture nucleic acid comprises SEQ ID NO: 22; the fifth capture nucleic acid comprises SEQ ID NO: 21; the sixth capture nucleic acid comprises SEQ ID NO: 23; the seventh capture nucleic acid comprises SEQ ID NO: 25; the eighth capture nucleic acid comprises SEQ ID NO: 26; the ninth capture nucleic acid comprises SEQ ID NO: 27; and the tenth capture nucleic acid comprises SEQ ID NO: 28. These sequences may be bound to a biotin moiety by a triethylene glycol linker.

Another aspect of the invention provides for a kit, in a suitable container, comprising a primer comprising between 6 to 30 nucleic acid bases complementary to the sequence of an RNA, which may be a mRNA. In some embodiments, the primer comprises between 6 to 30 nucleic acid bases complementary to the sequence adjacent to the 3′-end of the mRNA excluding the poly(A) tail. In some embodiments the mRNA is a hemoglobin chain mRNA. The kit may comprise a first primer comprising between 6 to 30 nucleic acid bases complementary to the contiguous 6, 7, 8, 9, 10, 11, 12, 13, 14, 15, 16, 17, 18, 19, 20, 21, 22, 23, 24, 25, 26, 27, 28, 29 or 30 nucleic acid bases at the 3′-end of SEQ ID NO: 1 or SEQ ID NO: 2; and a second primer comprising between 6 to 30 nucleic acid bases complementary to the contiguous 6, 7, 8, 9, 10, 11, 12, 13, 14, 15, 16, 17, 18, 19, 20, 21, 22, 23, 24, 25, 26, 27, 28, 29 or 30 nucleic acid bases at the 3′-end of SEQ ID NO: 3.

The terms “depleting,” “preventing, “inhibiting,” “reducing,” or “isolating,” or any variation of these terms, when used in the claims and/or the specification includes any measurable decrease or complete depletion, prevention, reduction, isolation or inhibition to achieve a desired result. “Depleting,” and “preventing” does not require complete depletion of target nucleic acid or, e.g., complete prevention of amplification of a nucleic acid. Throughout this application, the term “about” is used to indicate that a value related to includes the standard deviation of error for the method being employed to determine the value.

The use of the word “a” or “an” when used in conjunction with the term “comprising” in the claims and/or the specification may mean “one,” but it is also consistent with the meaning of “one or more,” “at least one,” and “one or more than one.”

It is specifically contemplated that any embodiments described in the Examples section are included as an embodiment of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The following drawings form part of the present specification and are included to further demonstrate certain aspects of the present invention. The invention may be better understood by reference to one or more of these drawings in combination with the detailed description of specific embodiments presented herein.

FIG. 1. Depiction of method of excluding amplification of specific transcripts during an RNA amplification from whole blood total RNA.

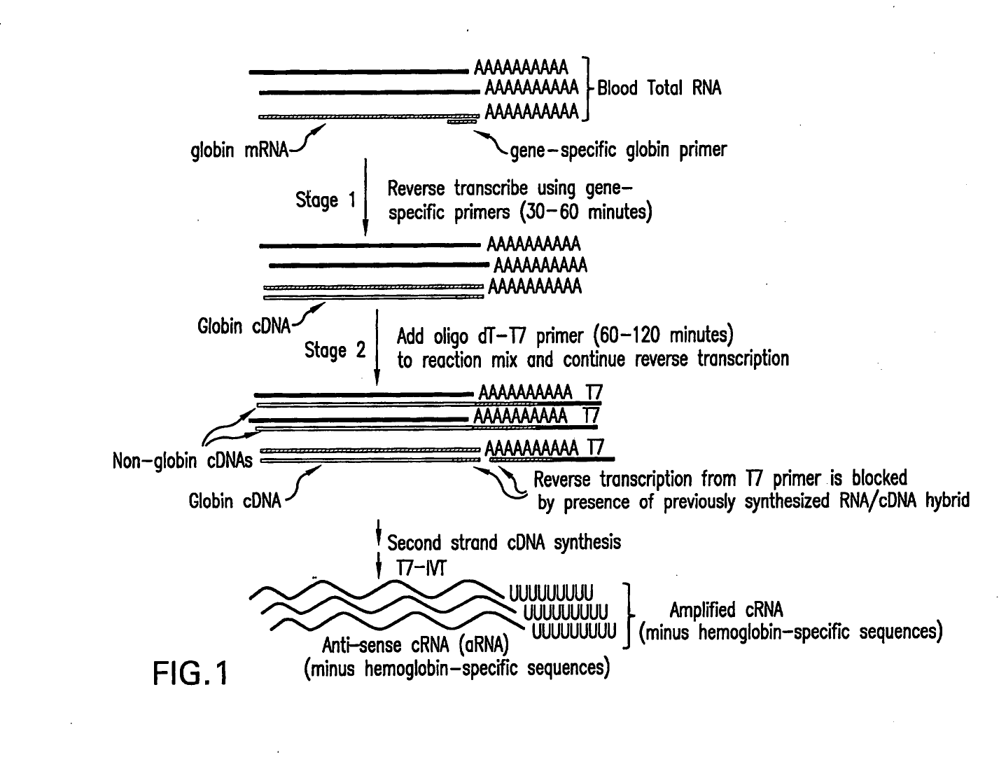

FIG. 2. Depiction of (a) method of capturing a mRNA transcript with a capture nucleic acid and a bridging nucleic acid and (b) method of capturing a mRNA transcript directly with a capture nucleic acid.

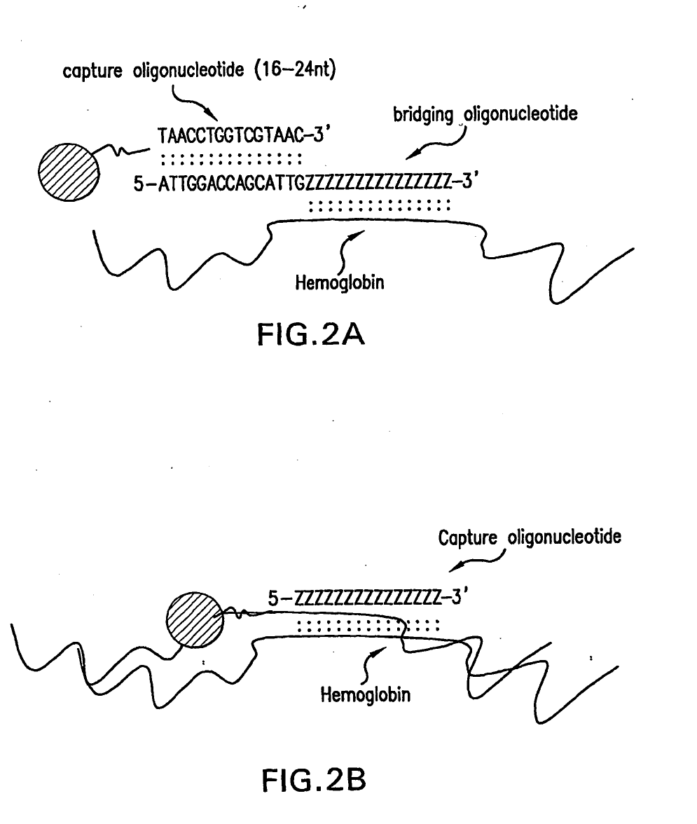

FIG. 3. Depiction of method of direct capturing of hemoglobin transcripts from the total RNA from whole blood using biotin and a streptavidin coated super-paramagnetic bead.

FIG. 4. Bioanalyzer trace of amplified RNA from both whole blood total RNA and the same whole blood RNA that has been processed by a direct capture method to remove the globin mRNA showing the complete disappearance of the prominent globin amplified RNA peak.

FIG. 5 GeneChip microarray comparison of total RNA samples where globin mRNA has been removed or unprocessed. Shown are 6 different donor blood samples. The number of genes called “Present” by the Affymetrix GCOS analysis are shown on the y-axis showing the increase in the number of genes that are shifted to a Present call after the globin mRNA is removed.

FIG. 6 Graphical representation of reduction in 3′-bias in beta actin during expression profiling by depletion of hemoglobin transcripts.

FIG. 7 Graphical representation of reduction in 3′-bias in GAPDH during expression profiling by depletion of hemoglobin transcripts.

FIG. 8 Bioanalyzer electropherograms of amplified total RNA from whole blood RNA, either untreated or blocked by globin specific primers. There is a complete disappearance of the “globin spike” with use of the globin-blocking primer oligonucleotides.

### DESCRIPTION OF ILLUSTRATIVE EMBODIMENTS

The present invention concerns a system for isolating, depleting, and/or preventing the amplification of specific, targeted nucleic acid populations, such as mRNA in a sample. The targeted nucleic acid, components of the system, and the methods for implementing the system, as well as variations thereof, are provided below.

### I. Targeted Nucleic Acid

The present invention concerns targeting a particular nucleic acid population (i.e., mRNA, rRNA, or tRNA) or targeting types of a nucleic acid population, such as individual mRNAs, tRNAs, rRNAs (e.g., 18S, or 28S). A nucleic acid is targeted by using a nucleic acid that has a targeting region—a region complementary to all or part of the targeted nucleic acid. In one aspect of the present invention, a primer comprises a targeting region. In another aspect of inventing, a capture nucleic acid, comprises the targeting region or a capture nucleic acid binds to a bridging nucleic acid that comprises the targeting region.

In some embodiments, the invention is specifically concerned with targeting mRNA, typically the targeted RNA is an abundant mRNA within a particular sample type. The sequences for mRNAs are well known to those of ordinary skill in the art and can be readily found in sequence databases such as GenBank (www.ncbi.nlm.nih.gov/) or are published. In embodiments wherein a primer comprises the targeting region for an mRNA, the primer typically binds at the 3′ of the transcript and adjacent to the 5′ end of the poly(A) tail. The target region complementary to the primer targeting region may range from 5 and up to 30 or from 5 up to 50 or more nucleotides in length. In some embodiments, the 3′ end of the target region complementary to the targeting region of the primer may be −1, −2, −3, −4, −5, −6, −7, −8, −10 bases in relation to the poly(A) tail, wherein −1 indicates the base immediately adjacent the 5′ end of the poly(A) tail. In other embodiments, the 3′ end of the target region complementary to the targeting region of the primer may be +1, +2, +3, +4 or +5 bases in relation to the poly(A) tail, wherein +1 indicates the first base of the poly(A) tail. In other embodiments, the 3′-end of the target region complementary to the targeting region of the primer may be in the range of −5 to −1, or −10 to −1, or −20 to −1, or −30 to −1, or −10 to −5, or −20 to −5, or −30 to −5, or −5 to +5, or −10 to +5, or −20 to +5, or −30 to +5, or −10 to +5, or −20 to +5, or −30 to +5 in relation to the 5′-end of the poly(A) tail. The terms “binding adjacent to the 5′ end of the poly(A)” and “binding adjacent to the 3′ end of a mRNA transcript” and “adjacently” in this context means for the purposes of the invention wherein the 3′ end of the target region complementary to the targeting region of the primer is in the range of −30 to +10 in relation to the 5′ end of the poly(A) tail. In other embodiments, a plurality of primers bind at multiple sites along the sequence of the mRNA, which may include the untranslated 5′ region, untranslated 3′ region, coding region, or may span such regions.

In another aspect of the invention, a capture nucleic acid comprises the region targeting an mRNA or a capture nucleic acid binds to a bridging nucleic acid that comprises the region targeting a mRNA. Embodiments include targeting regions that are complementary to all or part of the target mRNA, including all or part of the 5′-untranslated region, the 3′-untranslated region, or the coding region. In some embodiments, any region of at least five contiguous nucleotides in the targeted mRNA may be used as the targeted region—that is, the region that is complementary to the targeting region of a capture nucleic acid or a bridging nucleic acid. Also, there may be more than one targeted region in a mRNA. In some embodiments, there may be 1, 2, 3, 4, 5, or more targeted regions in a targeted mRNA. In some embodiments, the targeted region from a targeted mRNA acid is identical to a sequence in a different targeted nucleic acid. For example, the 3′-terminal 30 bases from both the 3′-untranslated region of human hemoglobin alpha 1 mRNA and the 3′-untranslated region of human hemoglobin alpha 2. are the same. Alternatively, a targeted region may be a sequence unique to a particular targeted nucleic acid. In some embodiments, the targeted region may be at least, or be at most 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more nucleotides in length.

In one aspect, the invention is concerned with targeting non-coding RNAs, such as rRNA or tRNA. Thus, e.g., the 18S, and/or 28S rRNA may be the targeted nucleic acid. The sequences for ribosomal RNAs are well known to those of ordinary skill in the art and can be readily found in sequence databases such as GenBank (www.ncbi.nlm.nih.gov/) or are published. In embodiments Wherein a primer comprises the targeting region, the target region complementary to the primer targeting region may range from 5 to 30 or may be 5 to 50 or more 50 nucleotides in length. Also, there may be more than one targeted region in a targeted non-coding RNA. There may be 1, 2, 3, 4, 5, 6, 7, 8, 9, 10 or more targeted regions in a targeted RNA. In another aspect of the invention, a capture oligonucleotide comprises the region targeting a non-coding RNA or a capture poligonulceotide binds to a bridging nucleic acid that comprises the region targeting a non-coding RNA. In another aspect of the invention, a capture oligonucleotide comprises the region targeting an non-coding RNA or a capture poligonulceotide binds to a bridging nucleic acid that comprises the region targeting a non-coding RNA. Non-coding RNAs may be targeted by targeting regions that are complementary to all or part of the non-coding RNA. Targeted non-coding RNAs may be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more nucleotides in length. Furthermore, any region of at least five contiguous nucleotides in the targeted non-coding RNA may be used as the targeted region—that is, the region that is complementary to the targeting region of a bridging nucleic acid. In one aspect the targeting region of a capture nor bridging nucleic acid is comprised of an in vitro synthesized complementary RNA transcript that transcript may contain one or more biotin moieties. In various embodiments biotin is incorporated into a transcript by nucleotide incorporation of modified NTPs containing biotin, end labeling, amino allyl reactive NTPs followed by chemical coupling with NHS esters of biotin. Also, there may be more than one targeted region in a targeted non-coding RNA. There may be 1, 2, 3, 4, 5, 6, 7, 8, 9, 10 or more targeted regions in a targeted non-coding RNA. A targeted region may be a region in a targeted non-coding RNA that has greater than 70%, 80%, or 90% homology with a sequence from a different targeted nucleic acid. In some embodiments, the targeted region from a targeted nucleic acid is identical to a sequence in a different targeted non-coding RNA. Alternatively, a targeted region may be a sequence unique to a particular targeted non-coding RNA.

Additional information regarding targeted nucleic acids is provided below. This information is provided as an example of targeted nucleic acid. However, it is contemplated that there may be sequence variations from individual organism to organism and these sequences provided as simply an example of one sequenced nucleic acid, even though such variations exist in nature. It is contemplated that these variations may also be targeted, and this may or may not require changes to a targeting nucleic acid or to the hybridization conditions, depending on the variation, which one of ordinary skill in the art could evaluate and determine.

A number of patents concern a targeted nucleic acid, for example, U.S. Pat. Nos. 4,486,539; 4,563,419; 4,751,177; 4,868,105; 5,200,314; 5,273,882; 5,288,609; 5,457,025; 5,500,356; 5,589,335; 5,702,896; 5,714,324; 5,723,597; 5,759,777; 5,897,783; 6,013,440; 6,060,246; 6,090,548; 6,110,678; 6,203,978; 6,221,581; 6,228,580; U.S. Patent Publication No. 20030175709 and WO 01/32672, all of which are specifically incorporated herein by reference.

A. mRNA

Typical targeted mRNAs of the invention are those that in a particular sample type, are present in an abundant amount. This is exemplified by the presence hemoglobin mRNAs in blood samples. The following examples of hemoglobin mRNA are provided, but the invention is not limited solely to these organisms and sequences (GenBank accession number provided):

| | | |
|---|---|---|
| 1. | Human | |
| | alpha 1 chain (HBA1) | NM_00558.3 |
| | alpha 2 chain (HBA2) | NM_00517.3 |
| | beta (HBB) | NM_00518.4 |
| | delta (HBD) | NM_000519.2 |
| | gamma A (HBG1) | NM_000559 |
| | gamma G (HBG2) | NM_000184 |
| 2. | Mouse | |
| | Adult chain 1 (Hba-a1) | NM_008218.1 |
| | Beta adult major chain | NM_008220.2 |
| 3. | Rat | |
| | Adult chain 1 (Hba-a1) | NM_013096 |
| | Beta chain cmples (Hbb) | NM_033234 |

Examples of other target mRNAs include:

| | |
|---|---|
| Ribosomal protein S3A | NM_001006 |
| Ribosomal protein L13 | NM_033251 |
| Ribosomal protein L32 | NM_001007073 |
| | NM_001007074 |
| Large ribosomal protein P0 | NM_053275 |
| Large ribosomal protein P1 | NM_213725 |
| GNAS Complex | NM_016592 |
| | NM_080425 |
| | NM_080426 |
| Tubulin, alpha 3 | NM_006082 |

B. Eukaryotic rRNA

Targeted nucleic acids of the invention may also be one or more types of eukaryotic rRNAs. Eukaryotes include, but are not limited to: mammals, fish, birds, amphibians, fungi, and plants. The following provides sequences for some of these targeted nucleic acids. It is contemplated that other eukaryotic rRNA sequences can be readily obtained by one of ordinary skill in the art, and thus, the invention includes, but is not limited to, the sequences shown below.

Superkingdom Eukaryota (eucaryotes)
Homo sapiens (human)

| | |
|---|---|
| 18S | M10098 |
| 18S | K03432 |
| 18S | X03205 |
| 28S | M11167 |

Mus muculus

| | |
|---|---|
| 18S | X00686 |
| 28S | X00525 |

Rattus norvegicus

| | |
|---|---|
| 18S | M11188 |
| 18S | X01117 |

Rattus norvegicus V01270.1

| | |
|---|---|
| 18S | 1-1874 |
| 28S | 3862-8647 |

C. tRNA

Targeted nucleic acids of the invention may also be one or more type of tRNA. In regard to targeting tRNAs, the secondary cloverleaf structure and the L-shaped tertiary structure limit the accessibility of complementary oligonucleotides to specific regions (Uhlenbeck, 1972; Schimmel et al. 1972; Freier. & Tinoco, 1975). These accessible regions include the NCCA sequence at the 3′-end, the anticodon loop, a portion of the D-loop, and a portion of the variable loop. The following examples of human tRNAs are provided, but the invention is not limited solely to this species and sequences (GenBank accession number provided):

| | |
|---|---|
| Ala tRNA | M17881 |
| Asn tRNA | K00167 |
| Leu tRNA | X04700 |
| Met tRNA | X04547 |
| Phe tRNA | K00350 |
| Ser tRNA | M27316 |
| Gly tRNA | K00209 |

### II. Primers

The present invention concerns compositions comprising a nucleic acid or a nucleic acid analog in a system or kit to prevent the amplification of a specific RNA or RNA population from other nucleic acids or nucleic acid populations, for which enrichment may be desirable. The term “primer” refers to a single-stranded oligonucleotide defined as being “extendable,” i.e., contains a free 3′ OH group that is available and capable of acting as a point of initiation for template-directed extension or amplification under suitable conditions, e.g., buffer and temperature, in the presence of four different nucleoside triphosphates and an agent for polymerization, such as, for example, reverse transcriptase. The length of the primer, in any given case depends on, for example, the intended use of the primer, and generally ranges from 3 to 6 and up to 30 or 50 nucleotides. Short primer molecules generally require cooler temperatures to form sufficiently stable hybrid complexes with the template. In some embodiments, the Tm's of the primers may range between 15-70° C., but typically have a Tm that is about 5° C. below that of the temperature utilized with the enzyme being used for reverse transcription (e.g., typically 37-50° C.). A primer needs not reflect the exact sequence of the template but must be sufficiently complementary to hybridize with such template. The targeted primer site is the area of the template to which a primer hybridizes. Primers can be DNA, RNA or comprise PNA or LNA and may be hybrids of DNA/LNA, DNA/PNA, DNA/RNA or combinations thereof. In some embodiments, a DNA/LNA has at least 2 modified LNA nucleotides in a DNA/LNA hybrid.

### III. Isolation and/or Depletion System Nucleic Acids

The present invention concerns compositions comprising a nucleic acid or a nucleic acid analog in a system or kit to deplete, isolate, or separate a nucleic acid population from other nucleic acid populations, for which enrichment may be desirable. It concerns either (1) direct capture wherein a capture nucleic acid comprises a targeting region, or (2) indirect capture using a capture nucleic acid that binds to a bridging nucleic acid that comprising a targeting region to deplete, isolate, or separate out a targeted nucleic acid, as discussed above.

A. Direct Targeting Nucleic Acid

Direct capture nucleic acids of the invention comprise a targeting region and a non-reacting structure that allows the direct targeting nucleic acid and any specifically bound target nucleic acid to be isolated away from other nucleic acid populations. The direct capture nucleic acid may comprise RNA, DNA, PNA, LNA or hybrids or mixtures thereof, or other analogs. In some embodiments, the targeting region comprises a sequence that is complementary to at least five contiguous nucleotides in the capture nucleic acid.

A non-reacting structure is a compound or structure that will not react chemically with nucleic acids, and in some embodiments, with any molecule that may be in a sample. Non-reacting structures may comprise plastic, glass, teflon, silica, a magnet, a metal such as gold, carbon, cellulose, latex, polystyrene, and other synthetic polymers, nylon, cellulose, nitrocellulose, polymethacrylate, polyvinylchloride, styrene-divinylbenzene, or any chemically-modified plastic. They may also be porous or non-porous materials. The structure may also be a particle of any shape that allows the targeted nucleic acid to be isolated, depleted, or separated. It may be a sphere, such as a bead, or a rod, or a flat-shaped structure, such as a plate with wells. Also, it is contemplated that the structure may be isolated by physical means or electromagnetic means. For example, a magnetic field may be used to attract a non-reacting structure that includes a magnet. The magnetic field may be in a stand or it may simply be placed on the side of a tube with the sample and a capture nucleic acid that is magnetized. Examples of physical ways to separate nucleic acids with their specifically hybridizing compounds are well known to those of skill in the art. A basket or other filter means may be employed to separate the capture nucleic acid and its hybridizing compounds (direct and indirect). The non-reacting structure and sample with nucleic acids of the invention may be centrifuged, filtered, dialyzed, or captured (with a magnet). When the structure is centrifuged it may be pelleted or passed through a centrifugible filter apparatus. The structure may also be filtered, including filtration using a pressure-driven system. Many such structures are available commercially and may be utilized herewith. Other examples can be found in WO 86/05815, WO90/06045, U.S. Pat. No. 5,945,525, all of which are specifically incorporated by reference.

Synthetic plastic or glass beads may be employed in the context of the invention. Beads are also referred to as micro-particles in this context. The beads may be complexed with avidin or streptavidin and they may also be super-paramagnetic. A suitable streptavidin super-paramagnetic microparticle is Sera-Mag™, available from Seradyn (Indianapolis, Ind.). They are nominal 1 to 10 micron super-paramagnetic micro-particles of uniform size with covalently bound streptavidin. These particles are colloidally stable in the absence of a magnetic field. The particles comprise a carboxylate-modified polystyrene core coated with magnetite and encapsulated with a polymer coating with streptavidin is covalently to the surface. The complexed streptavidin can be used to capture biotin linked to the direct targeting nuclide, either before or after hybridization to target nucleic acid. In some embodiments, biotin is linked via a phosphate group to the 5′-end of the direct capture nucleic acid, in other embodiments may be linked by a suitable linking agent such as a triethylene glycol linker (TEG). Such biotin labels are readily prepared by reagent known in the art, such as biotin phosphoramide or biotin TEG phosphoramide. Alternatively, the direct capture nucleic acid can be attached to the beads directly through chemical coupling. The beads may be collected using gravity- or pressure-based systems and/or filtration devices. If the beads are magnetized, a magnet can be used to separate the beads from the rest of the sample. The magnet may be employed with a stand or a stick or other type of physical structure to facilitate isolation.

Cellulose is a structural polymer derived from vascular plants. Chemically, it is a linear polymer of the monosaccharide glucose, using β, 1-4 linkages. Cellulose can be provided commercially, including from the Whatman company, and can be chemically sheared or chemically modified to create preparations of a more fibrous or particulate nature. CF-1 cellulose from Whatman is an example that can be implemented in the present invention. The beads may also be agarose.

Other components include isolation apparatuses such as filtration devices, including spin filters or spin columns.

B. Indirect Capture

1. Bridging Nucleic Acids

Bridging nucleic acids of the invention comprise a bridging region and a targeting region. As discussed in other sections, the location of these regions may be throughout the molecule, which may be of a variety of lengths. The bridging nucleic acid may comprise RNA, DNA, PNA, LNA or mixtures thereof, or other analogs.

In some embodiments, the bridging region comprises a sequence that is complementary to at least five contiguous nucleotides in the capture nucleic acid. It is contemplated that this region may be a homogenous sequence, that is, have the same nucleotide repeated across its length, such as a repeat of A, C, G, T, or U residues. However, to avoid hybridizing with a poly-A tailed mRNA in a sample comprising eukaryotic nucleic acids, it is contemplated that most embodiments will not have a poly-U or poly-T bridging region when dealing with such samples having poly-A tailed RNA. In some embodiments, the bridging region is a poly-C region and the capture region is a poly-G region, or vice versa. In other embodiments, the bridging region will be a random sequence that is complementary to the capture region (or the capture region will be random and the bridging region will be complementary to it). In further embodiments, the bridging region will have a designed sequence that is not homopolymeric but that is complementary to the capture region or vice versa. Sequences may be determined empirically. In many embodiments, it is preferred that this will be a random sequence or a defined sequence that is not a homopolymer. Some sequences will be determined empirically during evaluation in the assay.

2. Capture Nucleic Acids

Target regions of the Capture nucleic acids of the invention comprise a capture region and a non-reacting structure that allows the capture nucleic acid, any molecules specifically binding or hybridizing to the capture nucleic acid, i.e. the target nucleic acid in direct capture and for indirect capture, molecules specifically binding or hybridizing to the bridging nucleic acid and specifically bound targeted nucleic acid, to be isolated away from other nucleic acid populations.

In some embodiments, the bridging region comprises a sequence that is complementary to at least five contiguous nucleotides in the capture nucleic acid. It is contemplated that that this region may be a homogenous sequence, that is, have the same nucleotide repeated across its length, such as a repeat of A, C, G, T, or U residues. However, to avoid hybridizing with a poly-A tailed mRNA in a sample comprising eukaryotic nucleic acids, it is contemplated that most embodiments will not have a poly-U or poly-T bridging region when dealing with such samples having poly-A tailed RNA. In some embodiments, the bridging region is a poly-C region and the capture region is a poly-G region, or vice versa. In other embodiments, the bridging region will be a random sequence that is complementary to the capture region (or the capture region will be random and the bridging region will be complementary to it). In further embodiments, the bridging region will have a designed sequence that is not homopolymeric but that is complementary to the capture region or vice versa. Sequences may be determined empirically. In many embodiments, it is preferred that this will be a random sequence or a defined sequence that is not a homopolymer. Some sequences will be determined empirically during evaluation in the assay.

The capture nucleic acid may comprise RNA, DNA, PNA, LNA or hybrids or mixtures thereof, or other analogs. However, in some embodiments for indirect capture, it is specifically contemplated to be homopolymeric (only one type of nucleotide residue in molecule, such as poly-C), though in other embodiments, such as direct capture, it is specifically contemplated not to be homopolymeric and be heteropolymeric.

The main requirement for bridging and capture nucleic acid sequences is that they are complementary to one another. The capture region may be a poly-pyrimidine or poly-purine region comprising at least 5 nucleic acid residues. In addition, it may be heteropolymeric, either a random sequence or a designed sequence that is complementary to the bridging region of the nucleic acid with which it should hybridize.

A non-reacting structure attached or linked to the capture nucleic acid is employed in a similar fashion to the direct targeting nucleic acid as described above.

C. Nucleic Acid Compositions

The nucleic acid compositions of the present invention include targeting regions that target both mRNA and non-coding RNA targets. Typical mRNA targets are abundant mRNAs found in a particular sample, an example being hemoglobin transcripts in samples prepared from whole blood. Human mRNA targets include hemoglobin alpha 1 chain mRNA (SEQ ID NO: 1), hemoglobin alpha 2 chain mRNA (SEQ ID NO 2) and hemoglobin beta chain (SEQ ID NO: 3). Other mRNA targets include:

actin beta mRNA, SEQ ID NO: 4;
actin gamma 1 mRNA, SEQ ID NO: 5;
calmodulin 2 (phosphorylase kinase, delta) mRNA, SEQ ID NO: 6;
cofilin 1 (non-muscle) mRNA, SEQ ID NO: 7;
eukaryotic translation elongation factor 1 alpha 1 mRNA, SEQ ID NO: 8;
eukaryotic translation elongation factor 1 gamma mRNA, SEQ ID NO: 9;
ferritin, heavy polypeptide pseudogene 1 mRNA, SEQ ID NO: 10;
ferritin, light polypeptide mRNA, SEQ ID NO: 11;
glyceraldehyde-3-phosphate dehydrogenase mRNA, SEQ ID NO: 12;
GNAS complex locus mRNA, SEQ ID NO: 13;
translationally-controlled 1 tumor protein mRNA, SEQ ID NO: 14;
alpha 3 tubulin mRNA, SEQ ID NO: 15;
tumor protein mRNA, SEQ ID NO: 16;
translationally-controlled 1 mRNA, SEQ ID NO: 17; and
ubiquitin B mRNA, or ubiquitin C mRNA. SEQ ID NO: 18.

Other abundant mRNA targets include mRNA that encode ribosomal proteins, such as:

large ribosomal protein P0, SEQ ID NO: 29 mRNA;
large ribosomal protein P1, SEQ ID NO: 30 mRNA;
ribosomal protein S2, SEQ ID NO: 31 mRNA;
ribosomal protein S3A, SEQ ID NO: 32 mRNA;
ribosomal protein S4, SEQ ID NO: 33 mRNA;
ribosomal protein S6, SEQ ID NO: 34 mRNA;
ribosomal protein S10, SEQ ID NO: 35; mRNA
ribosomal protein S11, SEQ ID NO: 36; mRNA
ribosomal protein S13, SEQ ID NO: 37 mRNA;
ribosomal protein S14, SEQ ID NO: 38 mRNA;
ribosomal protein S15, SEQ ID NO: 39 mRNA;
ribosomal protein S18, SEQ ID NO: 40 mRNA
ribosomal protein S20, SEQ ID NO: 41 mRNA;
ribosomal protein S23, SEQ ID NO: 42; mRNA
ribosomal protein S27 (metallopanstimulin 1), SEQ ID NO: 43 mRNA;
ribosomal protein S28, SEQ ID NO: 44 mRNA;
ribosomal protein L3, SEQ ID NO: 45 mRNA;
ribosomal protein L7, SEQ ID NO: 46 mRNA;
ribosomal protein L7a, SEQ ID NO: 47; mRNA
ribosomal protein L10, SEQ ID NO: 48; mRNA
ribosomal protein L13, SEQ ID NO: 49 mRNA;
ribosomal protein L13a, SEQ ID NO: 50; mRNA
ribosomal protein L23a, SEQ ID NO: 51; mRNA
ribosomal protein L27a, SEQ ID NO: 52 mRNA;
ribosomal protein L30, SEQ ID NO: 53 mRNA;
ribosomal protein L31, SEQ ID NO: 54 mRNA;
ribosomal protein L32, SEQ ID NO: 55; mRNA
ribosomal protein L37a, SEQ ID NO: 56 mRNA;
ribosomal protein L38, SEQ ID NO: 57 mRNA;
ribosomal protein L39, SEQ ID NO: 58 mRNA; and
ribosomal protein L41, SEQ ID NO: 59 mRNA.

The primers of the present invention, will in typical embodiments be from 5 to 30 bases and be complementary to a sequence adjacent to the 3′-end of the mRNA (excluding the poly(A) tail). In some embodiments, the primers will comprise the antisense sequence complementary to the contiguous 6, 7, 8, 9, 10, 11, 12, 13, 14, 15, 16, 17, 18, 19, 20, 21, 22, 23, 24, 25, 26, 27, 28, 29 or 30 nucleic acid bases at the 3′-end of SEQ ID NO: 1 through SEQ ID NO: 18 and SEQ ID NO: 29 through 59.

The targeting regions of capture or bridging oligonucleotides will, in typical embodiments, comprise a sequence of at least 5 bases complementary to a target region in SEQ ID NO: 1 through SEQ ID NO: 18. Examples of suitable targeting region sequences specific for SEQ ID NO: 1 include SEQ ID NO: 19 and 20. Examples of suitable targeting region sequences specific for SEQ ID NO: 2 include SEQ ID NO: 21 and 22. An examples of a suitable targeting region sequence specific for both SEQ ID NO: 1 and SEQ ID NO: 2 is SEQ ID NO: 23. Suitable targeting region sequences specific for SEQ ID NO: 3 include SEQ ID NO: 24 through SEQ ID NO 28.

Typical non-coding RNA targets are abundant non-coding RNA targets found in a sample. Typical embodiments include human 18S and 28S rRNA. Non-coding rRNA targets include human 18S rRNA, SEQ ID NO: 60, 28S rRNA, SEQ ID NO: 61 and 5.8S (SEQ ID NO: 62). Examples of primers that target SEQ ID NO: 60 include SEQ ID NO: 74, SEQ ID NO: 75, SEQ ID NO: 76 and SEQ ID NO: 77. In typical embodiments, multiple primers may be used. Pairs of primers may bind adjacent to each other, in this case the pair of primers SEQ ID NO 74 and SEQ ID NO: 75 and the pair of primers SEQ ID NO: 76 and SEQ ID NO: 77, in both cases will have one base separating the pair, e.g., SEQ ID NO 74 and SEQ ID NO:75, if both primers are annealed to SEQ ID NO: 60. Examples of primers that target SEQ ID NO: 61 are SEQ ID NO: 78 through SEQ ID NO: 83. Again, these primers have pairs that bind such that one base will separate the annealed primers, such pairs being: SEQ ID NO: 78 and SEQ ID NO: 79; SEQ ID NO: 80 and SEQ ID NO: 81; and SEQ ID NO: 82 and SEQ ID NO: 83. Examples of primers that target SEQ ID NO: 62 are SEQ ID NO: 84 and SEQ ID NO: 85. This pair of primers will also have one base between then if both are annealed to SEQ ID NO: 62.

Primers will typically comprise a sequence of 5 to 30 or 5 to 50 or more bases complementary to a sequence of equal length in SEQ ID NO: 60 or SEQ ID NO: 61, while targeting regions of capture or bridging oligonucleotides will typically have a sequence of at least 5 bases up to the full length of the target such as SEQ ID. NO: 60 or SEQ ID NO: 61.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an Uralic “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those of a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table of non-limiting, purine and pyrimidine derivatives and analogs is also provided herein below.

TABLE 1
Purine and Pyrimidine Derivatives or Analogs

| Abbr. | Modified base description |
|---|---|
| ac4c | 4-acetylcytidine |
| Chm5u | 5-(carboxyhydroxylmethyl) |
| | uridine |
| Cm | 2′-O-methylcytidine |
| Cmnm5s2u | 5-carboxymethylamino- |
| | methyl-2-thioridine |
| Cmnm5u | 5- |
| | carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2- |
| | methylthiopurine-6- |
| | yl)carbamoyl)threonine |
| Mt6a | N-((9-beta-D-ribofuranosylpurine-6-yl)N- |
| | methyl-carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, |
| | (acp3)u |

A nucleobase may be comprised of a nucleoside or nucleotide, using any chemical or natural synthesis method described herein or known to one of ordinary skill in the art.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar.

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a subsistent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a subsistent moiety, which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythropentofuranosyl nucleotides for enhanced nuclease resistance, binding affinity, and ability to activate RNase H; and U.S. Pat. No. 5,708,154 which describes RNA linked to a DNA to form a DNA-RNA hybrid. Other analogs that may be used with compositions of the invention include U.S. Pat. No. 5,216,141 (discussing oligonucleotide analogs containing sulfur linkages), U.S. Pat. No. 5,432,272 (concerning oligonucleotides having nucleotides with heterocyclic bases), and U.S. Pat. Nos. 6,001,983, 6,037,120, 6,140,496 (involving oligonucleotides with non-standard bases), all of which are incorporated by reference.

5. Polyether and Peptide Nucleic Acids and Locked Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amino and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety. PNA oligomers can be prepared following standard solid-phase synthesis protocols for peptides (Merrifield, 1963; Merrifield, 1986) using, for example, a (methylbenzhydryl)amine polystyrene resin as the solid support (Christensen et al., 1995; Norton et al., 1995; Haaima et al., 1996; Dueholm et al., 1994; Thomson et al., 1995). The scheme for protecting the amino groups of PNA monomers is usually based on either Boc or Fmoc chemistry. The postsynthetic modification of PNA typically uses coupling of a desired group to an introduced lysine or cysteine residue in the PNA. Amino acids can be coupled during solid-phase synthesis or compounds containing a carboxylic acid group can be attached to the exposed amino-terminal amine group to modify PNA oligomers. A bis-PNA is prepared in a continuous synthesis process by connecting two PNA segments via a flexible linker composed of multiple units of either 8-amino-3,6-dioxaoctanoic acid or 6-aminohexanoic acid (Egholm et al., 1995).

PNAs are charge-neutral compounds and hence have poor water solubility compared to DNA. Neutral PNA molecules have a tendency to aggregate to a degree that is dependent on the sequence of the oligomer. PNA solubility is also related to the length of the oligomer and purine:pyrimidine ratio. Some modifications, including the incorporation of positively charged lysine residues (carboxyl-terminal or backbone modification in place of glycine), have shown improvement as to solubility. Negative charges may also be introduced, especially for PNA-DNA chimeras, which will enhance the water solubility.

Another non-limiting example is a locked nucleic acid or “LNA.” An LNA monomer is a bicyclic compound that is structurally similar to RNA nucleosides. LNAs have a furanose conformation that is restricted by a methylene linker that connects the 2′-O position to the 4′-C position, as described in Koshkin et al, 1998a and 1998b and Wahlestedt et al., 2000. LNA and LNA analogs display very high duplex thermal stabilities with complementary DNA and RNA (Tm=+3 to +10° C.), stability towards 3′-exonucleolytic degradation and good solubility properties. LNAs and oligonucleotides than comprise LNAs are useful in a wide range of diagnostic and therapeutic applications. Among these are antisense applications, PCR applications, strand-displacement oligomers, and substrates for nucleic acid polymerases. Phosphorothioate-LNA and 2′-thio-LNAs analogs have been reported (Kumar et al., 1998). Preparation of Locked Nucleoside Analogs Containing Oligodeoxyribonucleotide Duplexes as substrates for nucleic acid polymerases has also been described (WO98/0914). One group has added an additional methylene group to the LNA 2′,4′-bridging group (e.g. 4′-CH₂—CH₂—O-2′), U.S. Patent Application Publication No.: US 2002/0147332.

6. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. No. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

7. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

8. Nucleic Acid Segments

In certain embodiments, the nucleic acid comprises a nucleic acid segment. As used herein, the term “nucleic acid segment,” are smaller fragments of a nucleic acid, such as for non-limiting example, those that correspond to targeted, targeting, bridging, and capture regions. Thus, a “nucleic acid segment” may comprise any part of a gene sequence, of from about 2 nucleotides to the full length of a targeted nucleic acid, capture nucleic acid, or bridging nucleic acid.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
   where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition.

9. Nucleic Acid Complements

The present invention also encompasses a nucleic acid that is complementary to a other nucleic acids of the invention and targeted nucleic acids. More specifically, a targeting region in a bridging nucleic acid is complementary to the targeted region of the targeted nucleic acid and a bridging region of the bridging nucleic acid is complementary to a capture region of a capture nucleic acid. In particular embodiments the invention encompasses a nucleic acid or a nucleic acid segment identical or complementary to all or part of the sequences set forth in SEQ ID NOS:1-73. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. Unless otherwise specified, a nucleic acid region is “complementary” to another nucleic acid region if there is at least 70, 80%, 90% or 100% Watson-Crick base-pairing (A:T or A:U, C:G) between or between at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more contiguous nucleic acid bases of the regions. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semi-consecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, and any range derivable therein, of the nucleobase sequence is capable of base-pairing with a single or double stranded nucleic acid molecule during hybridization, as described in the Examples. In certain embodiments, the term “complementary” refers to a nucleic acid that may hybridize to another nucleic acid strand or duplex under conditions described in the Examples, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

10. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. Alternatively, stringent conditions may be determined largely by temperature in the presence of a TMAC solution with a defined molarity such as 3M TMAC. For example, in 3 M TMAC, stringent conditions include the following: for complementary nucleic acids with a length of 15 bp, a temperature of 45° C. to 55° C.; for complementary nucleotides with a length of 27 bases, a temperature of 65° C. to 75° C.; and, for complementary nucleotides with a length of >200 nucleotides, a temperature of 90° C. to 95° C. The publication of Wood et al., 1985, which is specifically incorporated by reference, provides examples of these parameters. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

11. Oligonucleotide Synthesis

Oligonucleotide synthesis is performed according to standard methods. See, for example, Itakura and Riggs (1980). Additionally, U.S. Pat. No. 4,704,362; U.S. Pat. No. 5,221,619, U.S. Pat. No. 5,583,013 each describe various methods of preparing synthetic structural genes.

Oligonucleotide synthesis is well known to those of skill in the art. Various different mechanisms of oligonucleotide synthesis have been disclosed in for example, U.S. Pat. No. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978; Gillam et al., 1979). Under controlled conditions, polynucleotide phosphorylase adds predominantly a single nucleotide to a short oligodeoxynucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

12. Expression Vectors

Other ways of creating nucleic acids of the invention include the use of a recombinant vector created through the application of recombinant nucleic acid technology known to those of skill in the art or as described herein. A recombinant vector may comprise a bridging or capture nucleic acid, particularly one that is a polynucleotide, as opposed to an oligonucleotide. An expression vector can be used create nucleic acids that are lengthy, for example, containing multiple targeting regions or relatively lengthy targeting regions, such as those greater than 100 residues in length.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1994, both incorporated herein by reference).

The term “expression vector” refers to any type of genetic construct comprising a nucleic acid coding for a RNA capable of being transcribed. Expression vectors can contain a variety of “control sequences,” which refer to nucleic acid sequences necessary for the transcription and possibly translation of an operable linked coding sequence in a particular host cell. In addition to control sequences that govern transcription (promoters and enhancers) and translation, vectors and expression vectors may contain nucleic acid sequences that serve other functions as well that are well known to those of skill in the art, such as screenable and selectable markers, ribosome binding site, multiple cloning sites, splicing sites, poly A sequences, origins of replication, and other sequences that allow expression in different hosts.

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The nucleotide and protein, polypeptide and peptide sequences for various genes have been previously disclosed, and may be found at computerized databases known to those of ordinary skill in the art. For example, the nucleotide sequences of rRNAs of various organisms are readily available. One such database is the National Center for Biotechnology Information's Genbank and GenPept databases (http://www.ncbi.nlm.nih.gov/). The coding regions for all or part of these known genes may be amplified and/or expressed using the techniques disclosed herein or by any technique that would be know to those of ordinary skill in the art.

13. Nucleic Acid Arrays

Because the present invention provides efficient methods of enriching in mRNA, which can be used to make cDNA, the present invention extends to the use of cDNAs with arrays. The term “array” as used herein refers to a systematic arrangement of nucleic acid. For example, a cDNA population that is representative of a desired source (e.g., human adult brain) is divided up into the minimum number of pools in which a desired screening procedure can be utilized to detect a cDNA and which can be distributed into a single multi-well plate. Arrays may be of an aqueous suspension of a cDNA population obtainable from a desired mRNA source, comprising: a multi-well plate containing a plurality of individual wells, each individual well containing an aqueous suspension of a different content of a cDNA population. The cDNA population may include cDNA of a predetermined size. Furthermore, the cDNA population in all the wells of the plate may be representative of substantially all mRNAs of a predetermined size from a source. Examples of arrays, their uses, and implementation of them can be found in U.S. Pat. Nos. 6,329,209, 6,329,140, 6,324,479, 6,322,971, 6,316,193, 6,309,823, 5,412,087, 5,445,934, and 5,744,305, which are herein incorporated by reference.

The number of cDNA clones array on a plate may vary. For example, a population of cDNA from a desired source can have about 200,000-6,000,000 cDNAs, about 200,000-2,000,000, 300,000-700,000, about 400,000-600,000, or about 500,000 cDNAs, and combinations thereof. Such a population can be distributed into a small set of multi-well plates, such as a single 96-well plate or a single 384-well plate. For instance, when about 1000-10,000 cDNAs, preferably about 3,500-7,000, more preferably about 5,000, from a population are present in a single well of a 96-well or 384-well plate, PCR can be utilized to clone a single, target gene using a set of primers.

The term a “nucleic acid array” refers to a plurality of target elements, each target element comprising one or more nucleic acid molecules immobilized on one or more solid surfaces to which sample nucleic acids can be hybridized. The nucleic acids of a target element can contain sequence(s) from specific genes or clones, e.g. from the regions identified here. Other target elements will contain, for instance, reference sequences. Target elements of various dimensions can be used in the arrays of the invention. Generally, smaller, target elements are preferred. Typically, a target element will be less than about 1 cm in diameter. Generally element sizes are from 1 μm to about 3 mm, between about 5 μm and about 1 mm. The target elements of the arrays may be arranged on the solid surface at different densities. The target element densities will depend upon a number of factors, such as the nature of the label, the solid support, and the like. One of skill will recognize that each target element may comprise a mixture of nucleic acids of different lengths and sequences. Thus, for example, a target element may contain more than one copy of a cloned piece of DNA, and each copy may be broken into fragments of different lengths. The length and complexity of the nucleic acid fixed onto the target element is not critical to the invention. One of skill can adjust these factors to provide optimum hybridization and signal production for a given hybridization procedure, and to provide the required resolution among different genes or genomic locations. In various embodiments, target element sequences will have a complexity between about 1 kb and about 1 Mb, between about 10 kb to about 500 kb, between about 200 to about 500 kb, and from about 50 kb to about 150 kb.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

A microarray may contains binding sites for products of all or almost all genes in the target organism's genome, but such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to the action of a drug of interest or in a biological pathway of interest. A “gene” is identified as an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence.

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995a. See also DeRisi et al., 1996; Shalon et al., 1996; Schena et al., 1995b. Each of these articles is incorporated by reference in its entirety.

Other methods for making microarrays, e.g., by masking (Maskos et al., 1992), may also be used. In principal, any type of array, for example, dot blots on a nylon hybridization membrane (see Sambrook et al., 1989, which is incorporated in its entirety for all purposes), could be used, although, as will be recognized by those of skill in the art, very small arrays will be preferred because hybridization volumes will be smaller.

Labeled cDNA is prepared from mRNA by oligo dT-primed or random-primed reverse transcription, both of which are well known in the art (see e.g., Klug et al., 1987). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

Fluorescently-labeled probes can be used, including suitable fluorophores such as fluorescein, lissamine, phycoerythrin, rhodamine (Perkin Elmer Cetus), Cy2, Cy3, Cy3.5, Cy5, Cy5.5, Cy7, Fluor X (Amersham) and others (see, e.g., Kricka, 1992). It will be appreciated that pairs of fluorophores are chosen that have distinct emission spectra so that they can be easily distinguished. In another embodiment, a label other than a fluorescent label is used. For example, a radioactive label, or a pair of radioactive labels with distinct emission spectra, can be used (see Zhao et al., 1995; Pietu et al., 1996). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™, Invitrogen Inc.) at 42° C. for 60 min.

### IV. Methods for Depleting and Preventing Amplification of Targeted Nucleic Acids

Methods of the invention involve preparing a sample comprising a targeted nucleic acid, preparing a bridging nucleic acid, preparing a capture nucleic acid, incubating nucleic acids under conditions allowing for hybridization among complementary regions, washing the sample and/or the capture and/or bridging nucleic acids, and isolating the capture nucleic acids and any accompanying compounds (compounds that bind or hybridize directly or indirectly to the capture nucleic acids). Methods of the invention also involve preparing a primer that does not comprise a DNA polymerase promoter sequence, binding the primer to an RNA in an RNA sample, incubating the sample under conditions suitable for reverse transcription, adding a primer comprising a DNA polymerase promoter sequence, incubating the sample under conditions suitable for reverse transcription, degrading the RNA strand, incubating the sample under conditions for transcription of a second DNA strand to form a cDNA. Steps of the invention are not required to be in a particular order and thus, the invention covers methods in which the order of the steps varies.

Hybridization conditions are discussed earlier. Wash conditions may involve temperatures between 20° C. and 75° C., between 25° C. and 70° C., between 30° C. and 65° C., between 35° C. and 60° C., between 40° C. and 55° C., between 45° C. and 50° C., or at temperatures within the ranges specified.

Buffer conditions for hybridization of nucleic acid compositions are well known to those of skill in the art. It is specifically contemplated that isostabilizing agents may be employed in hybridization and wash buffers in methods of the invention. U.S. Ser. No. 09/854,412 describes the use of tetramethylammonium chloride (TMAC) and tetraethylammonium chloride (TEAC) in such buffers; this application is specifically incorporated by reference herein. The concentration of an isostabilizing agent in a hybridization (binding) buffer may be between about 1.0 M and about 5.0 M, is about 4.0 M, or is about 2.0 M. Also specifically contemplated is a wash solution with an isostabilizing agent concentration of between about 0.1 M and 3.0 M, including 0.1 M increments within the range. Wash buffers may or may not contain Tris. However, in some embodiments of the invention, the wash solution consists of water and no other salts or buffers. In some embodiments of the invention, the hybridizing or wash buffer may include guanidinium isothiocyanate, though in some embodiments this chemical is specifically contemplated to be absent. The concentration of guanidinium may be between about 0.4 M and about 3.0 M

A solution or buffer to elute targeted nucleic acids from the hybridizing nucleic acids (indirect or direct) may be implemented in some kits and methods of the invention. The elution buffer or solution can be an aqueous solution lacking salt, such as TE or water. Elution may occur at room temperature or it may occur at temperatures between 15° C. and 100° C., between 20° C. and 95° C., between 25° C. and 90° C., between 30° C. and 85° C., between 35° C. and 80° C., between 40° C. and 75° C., between 45° C. and 70° C., between 50° C. and 65° C., between 55° C. and 60° C., or at temperatures within the ranges specified.

A. Quantization of RNA

1. Assessing RNA Yield by UV Absorbance

The concentration and purity of RNA can be determined by diluting an aliquot of the preparation (usually a 1:50 to 1:100 dilution) in TE (10 mM Tris-HCl pH 8, 1 mM EDTA) or water, and reading the absorbance in a spectrophotometer at 260 nm and 280 nm.

An A₂₆₀ of 1 is equivalent to 40 μg RNA/ml. The concentration (μg/ml) of RNA is therefore calculated by multiplying the A₂₆₀ X dilution factor X 40 μg/ml. The following is a typical example:

The typical yield from 10 μg total RNA is 3-5 μg. If the sample is re-suspended in 25 μl, this means that the concentration will vary between 120 ng/μl and 200 ng/μl. One μl of the prep is diluted 1:50 into 49 μl of TE. The A₂₆₀=0.1. RNA concentration=0.1×50×40 μg/ml=200 μg/ml or 0.2 μg/μl. Since there are 24 μl of the prep remaining after using 1 μl to measure the concentration, the total amount of remaining RNA is 24 μl×0.2 μg/μl=4.8 μg.

2. Assessing RNA Yield with RiboGreen®

Molecular Probes' RiboGreen® fluorescence-based assay for RNA quantization can be employed to measure RNA concentration.

B. Denaturing Agarose Gel Electrophoresis

Many mRNAs form extensive secondary structure. Ribosomal RNA depletion may be evaluated by agarose gel electrophoresis. Because of this, it is best to use a denaturing gel system to analyze RNA samples. A positive control should be included on the gel so that any unusual results can be attributed to a problem with the gel or a problem with the RNA under analysis. RNA molecular weight markers, an RNA sample known to be intact, or both, can be used for this purpose. It is also a good idea to include a sample of the starting RNA that was used in the enrichment procedure.

Ambion's NorthernMax™ reagents for Northern Blotting include everything needed for denaturing agarose gel electrophoresis. These products are optimized for ease of use, safety, and low background, and they include detailed instructions for use. An alternative to using the NorthernMax reagents is to use a procedure described in “Current Protocols in Molecular Biology”, Section 4.9 (Ausubel et al., eds.), hereby incorporated by reference. It is more difficult and time-consuming than the Northern-Max method, but it gives similar results.

C. Agilent 2100 Bioanalyzer

1. Evaluating rRNA Removal with the RNA 6000 LabChip

An effective method for evaluating rRNA removal utilizes RNA analysis with the Caliper RNA 6000 LabChip Kit and the Agilent 2100 Bioanalayzer. Follow the instructions provided with the RNA 6000 LabChip Kit for RNA analysis. This system performs best with RNA solutions at concentrations between 50 and 250 ng/μl. Loading 1 μl of a typical enriched RNA sample is usually adequate for good performance.

2. Expected Results

In enriched human mRNA, the 18S and 28S rRNA peaks will be absent or present in only very small amounts. The peak calling feature of the software may fail to identify the peaks containing small quantities of leftover 16S and 23S rRNAs. A peak corresponding to 5S and tRNAs may be present depending on how the total RNA was initially purified. If RNA was purified by a glass fiber filter method prior to enrichment, this peak will be smaller. The size and shape of the 5S rRNA-tRNA peak is unchanged by some embodiments.

D. Reverse Transcription

The invention provides for reverse transcription of a first-strand cDNA using an abundant RNA as a template after binding of a primer that does not comprise a DNA polymerase promoter sequence. The primer is annealed to RNA forming a primer:RNA complex. Extension of the primer is catalyzed by reverse transcriptase, or by a DNA polymerase possessing reverse transcriptase activity, in the presence of adequate amounts of other components necessary to perform the reaction, for example, deoxyribonucleoside triphosphates dATP, dCTP, dGTP and dTTP, Mg²⁺, and optimal buffer. A variety of reverse transcriptases can be used. The reverse transcriptase may be Moloney murine leukemia virus (M-MLV) (U.S. Pat. No. 4,943,531) or M-MLV reverse transcriptase lacking RNaseH activity (U.S. Pat. No. 5,405,776), avian myeloblastosis virus (AMV). These reverse transcriptases may be an engineered version such a SuperScript® (I, II and III) or eAMV®.

cDNA is also prepared from mRNA by oligo dT-primed reverse transcription, both. The reaction is typically catalyzed by an enzyme from a retrovirus, which is competent to synthesize DNA from an RNA template. Generally the primer used for reverse transcription has two parts: one part for annealing to the RNA molecules in the cell sample through complementarity and a second part comprising a strong promoter sequence. Typically the strong promoter is from a bacteriophage, such as SP6, T7 or T3. Because most populations of mRNA from biological samples do not share any sequence homology other than a poly(dA) tract at the 3′ end, the first part of the primer typically comprises a poly(dT) sequence which is generally complementary to most mRNA species.

### V. KITS

Any of the compositions described herein may be comprised in a kit. In a non-limiting example, a bridging nucleic acid and a capture nucleic acid may be comprised in a kit; or one or more capture nucleic acids may be comprised in a kit, or one or more primers specific for an RNA may be comprised in a kit. The kits will thus comprise, in suitable container means, a the nucleic acids of the present invention. It may also include one or more buffers, such as hybridization buffer or a wash buffer, compounds for preparing the sample, and components for isolating the capture nucleic acid via the nonreacting structure. Other kits of the invention may include components for making a nucleic acid array, and thus, may include, for example, a solid support.

The kits may comprise suitably aliquoted nucleic acid compositions of the present invention, whether labeled or unlabeled, as may be used to isolate, deplete, or prevent the amplification of a targeted nucleic acid. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the targeting molecule, such as filters, beads, or a magnetic stand. Such kits generally will comprise, in suitable means, distinct containers for each individual reagent or solution as well as for the targeting agent.

A kit will also include instructions for employing the kit components as well the use of any other reagent not included in the kit. Instructions may include variations that can be implemented.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Furthermore, these examples are provided as one of many ways of implementing the claimed method and using the compositions of the invention. It is contemplated that the invention is not limited to the specific conditions set forth below, but that the conditions below provide examples of how to implement the invention.

### Example 1

### Materials

The following materials were used in the methods described herein for the selective removal of hemoglobin transcripts by capture nucleic acids from total RNA from whole blood.

Globin Capture Oligo Mix: 1-10 M final concentration of capture oligos should be diluted in 10 mM Tris HCl 0.1 mM EDTA ph 8.0. There are 10 capture oligos in the mix, each one at 1-10 μM. All oligos have a 5′ TEG-Biotin modification. All oligos were HPLC purified: Oligos were 5BioTEG/ctccagggcctccgcaccatactc; 5BioTEG/tggtggtggggaaggacaggaaca; 5BioTEG/ggtcgaagtgcgggaagtaggtct; 5BioTEG/gtcagcgcgtcggccaccttctt; 5BioTEG/ctccagggcctccgcaccatactc; 5BioTEG/gccgcccactcagactttattcaa; 5BioTEG/ccacagggcagtaacggcagac; 5BioTEG/cataacagcatcaggagtggacaga; 5BioTEG/ccatcactaaaggcaccgagcact; 5BioTEG/cattagccacaccagccaccactt; and 5BioTEG/ggcccttcataatatcccccagtt.

2× Hybridization Buffer: For a 1 liter batch combine: 600 ml 5M-15M TEMAC, 100 ml 0.1M-1M Tris-HCl pH 8.0, 50 ml 0.02M-0.5M EDTA pH 8.0, 100 ml 1%-10% SDS and 150 ml Nuclease-Free Water

Streptavidin Bead Buffer: For a 1 liter batch combine: 300 ml 5M-15M TEMAC, 50 ml 0.2M-1M Tris-HCl pH 8.0, 25 ml 0.5M EDTA pH 8.0, 50 ml 1%-10% SDS and 575 ml Nuclease-Free Water.

### Example 2

### Removal of Alpha and Beta Hemoglobin mRNA by Capture Nucleic Acids from Total RNA Prepared from Human Blood

### 1. Isolation of Total RNA

Total RNA was isolated from whole blood using RiboPure-Blood™ Kit (Ambion), following the instructions as supplied with the kit.

### 2. RNA Precipitation

The following reagents were added to each RNA sample and mixed thoroughly: 0.1 vol. of 5 M ammonium acetate or 3 M sodium acetate; 5 μg glycogen; and 2.5-3 vol. 100% ethanol. The glycogen is optional and acts as a carrier to improve the precipitation for solutions with less than 200 μg RNA/ml. The mixture was placed at −20° C. overnight. Alternative procedures utilized were quick freezing in ethanol and dry ice or in a −70° C. freezer for 30 min. The mixture was then centrifuged at 12,000×g for 30 min. at 40 to recover the RNA. The supernatant was carefully removed and discarded. Ice cold 70% ethanol (1 ml) was added to the mixture and vortexed. The RNA was re-pelleted by centrifuging for 10 min. at 4° C. and the supernatant was again carefully removed and discarded. The samples were rewashed in ice cold 70% ethanol using the same procedure. The RNA sample was resuspended in <14 μl 10 mM Tris-HCl pH 8, 1 mM EDTA.

3. Removal of Hemoglobin mRNA

Removal of alpha and beta hemoglobin mRNA was removed using a Globin mRNA Removal Kit. Materials provided with the kit include reagents for depletion of hemoglobin mRNA and also for mRNA purification. The hemoglobin mRNA depletion reagents supplied are: 1.5 ml of 2× hybridization buffer; 1.5 ml streptavidin bead buffer, 600 μl streptavidin super-paramagnetic beads; 20 μl capture oligo mix; and 1.75 ml nuclease-free water.

The 2× hybridization buffer and the streptavidin bead buffer were warmed to 50° C. for 15 min. and vortexed well before use. The streptavidin super-paramagnetic beads were vortexed to suspend the beads, and volume transferred to 1.5 ml tube sufficient for 301 added to each sample tube. The beads were collected by briefly centrifuged (<2 sec.) the 1.5 ml tube at a low speed (<1000×g). The tube was left on a magnetic stand to capture the streptavidin super-paramagnetic beads until the mixture because transparent, indicating that the capture was completed. The supernatant was carefully removed and discarded and the tube removed from the magnetic stand. The streptavidin bead buffer was added to the streptavidin beads, using a volume equal to the original volume of streptavidin beads, and vortexed vigorously until the beads were resuspended, and then placed at 50° C.

The following were combined in a 1.5 ml non-stick tube: 1-10 μg human whole blood total RNA; and 1 μl of capture oligo mix. Nuclease-free water was added to samples to a volume of 15 μl when necessary and then 15 μl of the 50° C. 2× hybridization buffer, and then vortexed briefly followed by centrifugation briefly and the contents collected in the bottom of the tube. The samples were incubated at 50° C. for 15 minutes to allow the capture oligo mix to the hemoglobin mRNA.

The pre-prepared streptavidin beads preheated to 50° C. were resuspended by gentle vortexing and 30 μl was added to each RNA sample. The mixtures were incubated at 50° C. for 30 min. Samples were then placed on a magnetic stand until the mixtures became transparent indicating that the beads had been captured. The supernatant containing the RNA was transferred to a new 1.5 ml tube.

The RNA was purified using the kit reagents: 200 μl RNA binding beads, 80 μl RNA bead buffer; 4 ml RNA binding buffer concentrate with 4 ml of 100% ethanol added before use; 5 ml RNA wash solution concentrate with 4 ml 100% ethanol added before use; and 1 ml elution buffer. To each enriched RNA sample was added 100 μl prepared RNA binding buffer and them 20 μl of RNA binding beads prepared by concentrating the stock on a magnetic stand and washing the beads with 20 μl of vortexed bead resuspension mix prepared by adding RNA binding buffer (10 μl per sample) and RNA bead buffer (4 μl per sample), mix briefly and add 100% isopropanol (6 μl per sample). Samples were vortexed for 10 sec. to fully mix the reagents and allow the RNA binding beads to bind the RNA. Samples were briefly centrifuged (<2 sec.) at low speed (<1000×g) then ten placed on a magnetic stand to capture the super-paramagnetic beads, indicated by the mixture becoming transparent. The supernatant was aspirated and discarded. The sample was removed from the magnetic stand and 200 μl RNA wash solution was added and vortexed for 10 sec. Samples were briefly centrifuged (<2 sec.) at low speed (<1000×g) and the capture procedure repeated. Samples were air dried for 5 min. after the supernatants were aspirated and discarded. To each sample was added 30 μl of elution buffer prewarmed to 58° C. and vortexed vigorously for about 10 sec. The RNA beads were captured using a magnetic stand and the supernatants containing the RNA stored at −20° C.

### Example 3

### Comparison of mRNA with and without Removal of Alpha and Beta Hemoglobin mRNA by Capture Nucleic Acids

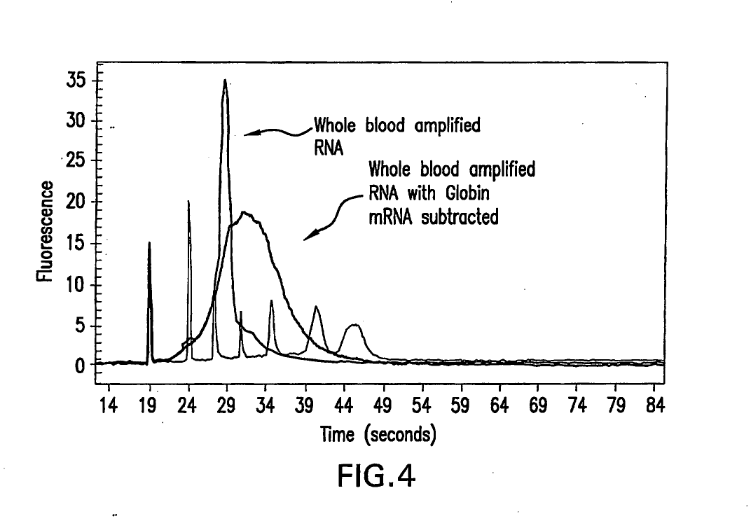

Both 1 μg RNA and μg enriched RNA were linearly amplified using the MessageAmp™ II Kit (Ambion) as per the supplied instructions. The resulting aRNA was run on an Agilent 2100 bioabalyzer RNA LabChip assay to compare the aRNA samples. The results are shown in FIG. 4. The disappearance of the distinctive hemoglobin aRNA peak in the enriched RNA is clearly notable.

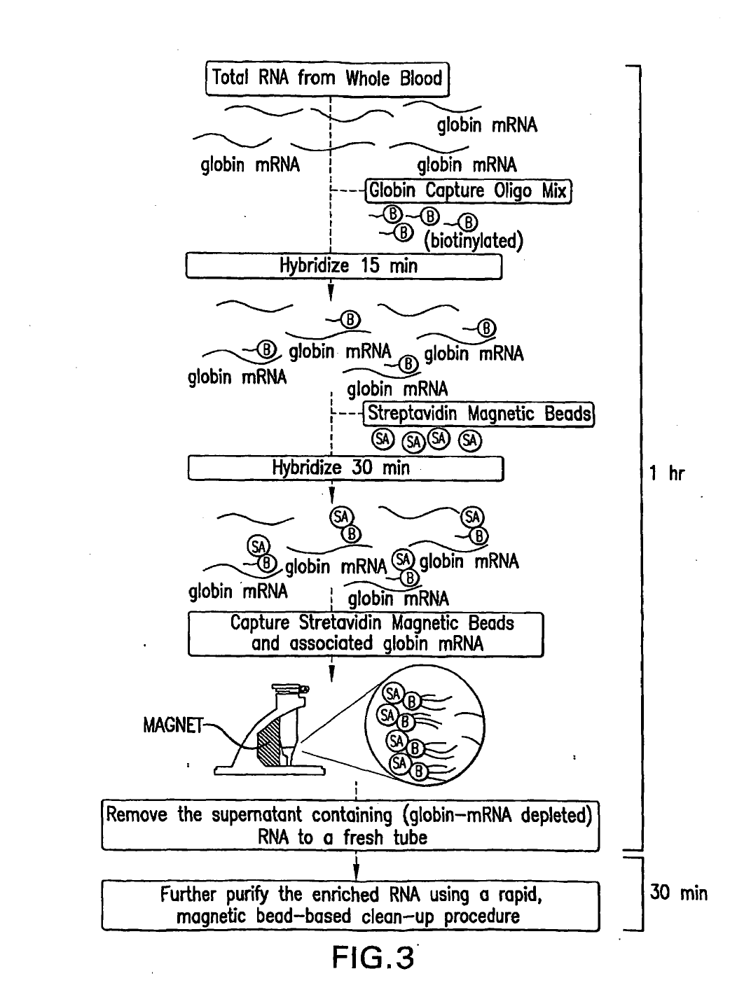

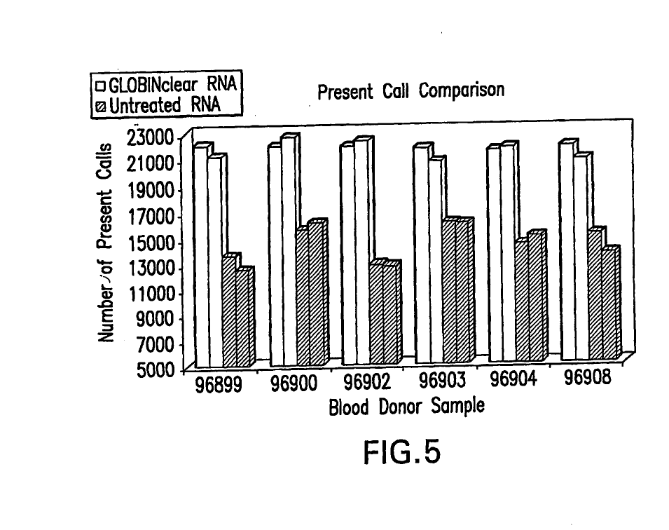

Results of a comparison of samples from 6 donors analyzed by Affymetrix GeneChip microarray is shown in FIG. 5. The number of genes called “present” by the Affymetrix GCOS analysis are shown in the y-axis. There is a notable number in the genes called Present after the globin mRNA has been removed. The extent of removal of the alpha and beta globin mRNAs in the 6 sets of donor samples, i.e., total RNA and enriched RNA, was investigated by qRT-PCR. The results, summarized in FIG. 3E, shows the fold reduction of the mRNAs of the two globin chains in the enriched RNA samples as compared to total RNA samples.

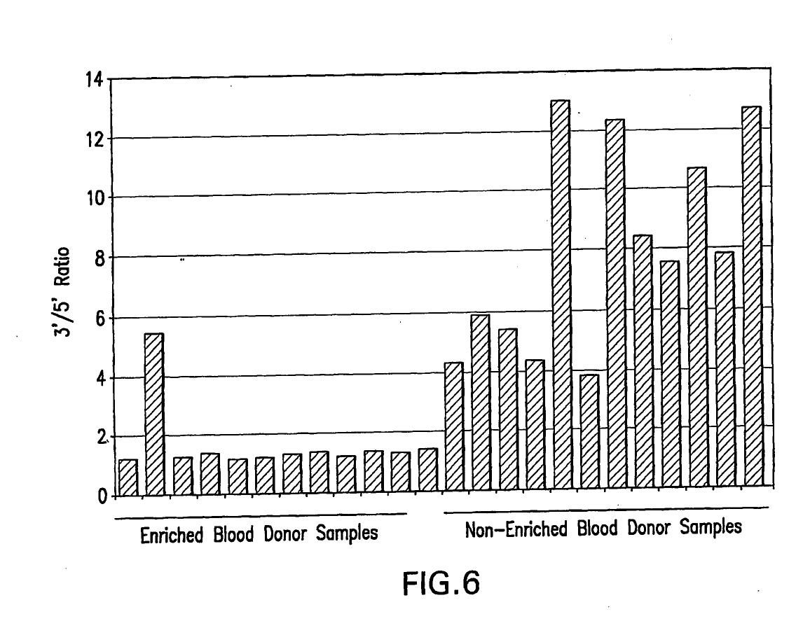

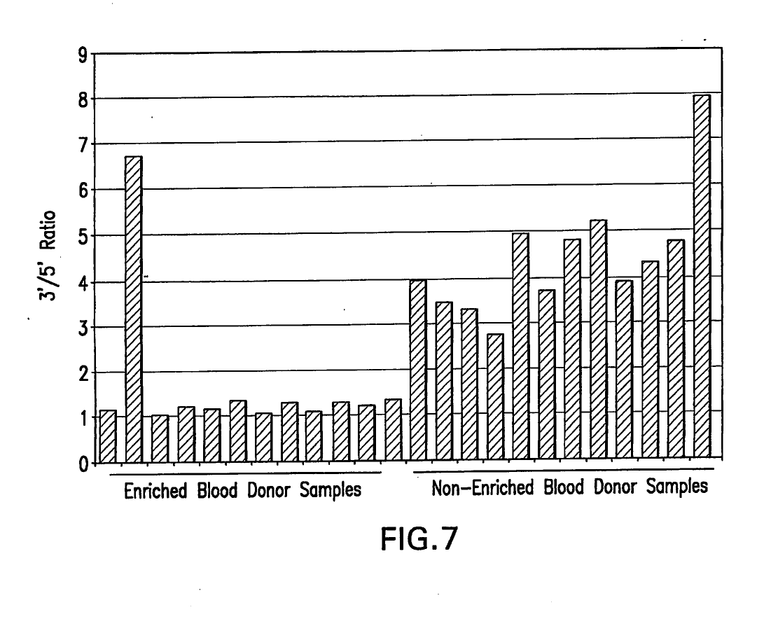

Depletion of globin mRNA also reduced the 3′ bias during expression profiling, as shown by analysis of actin and glyceraldehyde-3-phosphate dehydrogenase (GAPDH) 3′/5′ signal ratios. The 3′/5′ signal ratios were examined by comparing the hybridization signal intensity of probe sets interrogating the 3′ and 5′ ends of the actin and GAPDH transcript. The results, shown in FIG. 6 and FIG. 7, clearly indicate that removal of the alpha and beta globin mRNAs generally virtually eliminates the 3′ bias.

### Example 4

### Removal of Alpha and Beta Globin mRNA from Total RNA Prepared from Human Blood by use of Globin Specific Primers

ArrayScript™ (Ambion) is a rationally engineered version of the wild-type M-MLV reverse transcriptase such that the modified enzyme. This and other reagents are from the MessageAmp™ II aRNA Amplification Kit (Ambion).

Primers directed at the 3′ end of globin alpha chain mRNAs were:

| | |
|---|---|
| 5′-GCCGCCCACTCAGACTTTATT-3′ | (SEQ ID NO: 63) |
| 5′-AAAGACCACGGGGGTA-3′ | (SEQ ID NO: 64) |
| 5′-CCACTCAGACTT-3′ | (SEQ ID NO: 65) |
| 5′-AAAGACCACGG-3′ | (SEQ ID NO: 66) |
| 5′-CCACTCAGACTT-3′ | (SEQ ID NO: 67) |
| 5′-AAAGACCACGG-3′ | (SEQ ID NO: 68) |

Primers directed at the 3′ end of globin beta chain mRNAs were:

| | |
|---|---|
| 5′-GCAATGAAAATAAATG-3′ | (SEQ ID NO: 69) |
| 5′-TTTATTAGGCAGAATCCAGATG-3′ | (SEQ ID NO: 70) |
| 5′-TTTATTAGGCAGAAT-3′ | (SEQ ID NO: 71) |
| 5′- AATGAAAATAAATG-3′ | (SEQ ID NO: 72) |
| 5′-TTTATTAGGCAGAAT-3′ | (SEQ ID NO: 73) |

Bold and underlined bases indicated LNA modified bases

### 1. Preparation of Whole Blood RNA

RNA samples were prepared as described previously in Example 2.

2. Removal of Hemoglobin mRNA

A) LNA Annealing Setup.

| | | |
|---|---|---|
| Blood Total RNA | 1 | ug |
| Alpha & Beta Globin specific LNA mix (10 pmol/ul)) | 1.0 | ul |
| Nuclease Free Water | x | ul |
| Total Volume | 6.0 | ul |

Incubate at 70° C. for 10 minutes.

B) Extension Reaction Setup

After annealing the LNAs to the same tube add:

| | |
|---|---|
| 10x ArrayScript RT buffer | 1.0 ul |
| dNTP mix | 2.0 ul |
| Ribonuclease Inhibitor Protein | 0.5 ul |
| ArrayScript Reverse Transcriptase | 0.5 ul |
| Total Volume | 10.0 ul |

Incubate at 48° C. for 20 minutes.

C) T7dT Annealing and RT Set-Up of Poly A RNA

To the reaction add:

| | |
|---|---|
| T7oligodT (6 pmol/ul) | 1.0 ul |
| 10x ArrayScript RT buffer | 1.0 ul |
| dNTP mix | 2.0 ul |
| Ribonuclease Inhibitor Protein | 0.5 ul |
| ArrayScript Reverse Transcriptase | 0.5 ul |
| Nuclease Free water | 5.0 ul |
| Final Volume | 20.0 ul |

Incubate at 42° C. for 2 hours.

Second strand synthesis, ds cDNA purification and in vitro transcription were conducted as provided for by MessageAmp™ II aRNA Amplification Kit (Ambion) and as briefly described below:

D) Second Strand cDNA Synthesis
1. Add 80 μl Second Strand Matter Mix to each samples

E) cDNA Purification
1. Preheat Nuclease-free Water to 50-55° C.
2. Add 250 μl cDNA Binding Buffer to each sample
3. Pass the mixture through a cDNA Filter Cartridge
4. Wash with 500 μl Wash Buffer
5. Elute cDNA with 2×10 μl 50-55° C. Nuclease-free Water

F) In Vitro Transcription to Synthesize aRNA
1. Mix biotin NTPs with the cDNA and concentrate
2. Add IVT Master Mix to each sample
3. Incubate for 4-14 hr at 37° C.
4. Add Nuclease-free Water to bring each sample to 100 μl

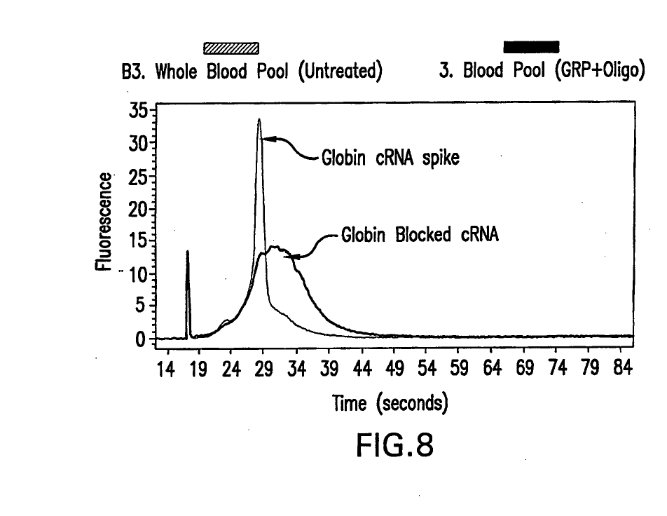

G) aRNA Purification
1. Preheat Nuclease-free Water to 50-60° C. (>10 min)
2. Assemble aRNA Filter Cartridge and tubes
3. Add 350 μl aRNA Binding Buffer
4. Add 250 μl 100% ethanol and pipet 3 times to mix
5. Pass samples through an a RNA Filter Cartridge(s)
6. Wash with 650 μl Wash Buffer
7. Elute aRNA with 100 μl preheated Nuclease-free Water
8. Store aRNA at −80° C.
   Bioanalyzer electropherograms of amplified total RNA from whole blood RNA, either untreated or blocked with the globin specific primers is shown in FIG. 8. There is a complete disappearance of the “globin spike” with use of the globin blocking primer oligonucleotides.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. application Ser. No. 09/854,412
US Application Publication No. 2002/0147332
U.S. Pat. No. 4,486,539
U.S. Pat. No. 4,563,419
U.S. Pat. No. 4,659,774
U.S. Pat. No. 4,682,195
U.S. Pat. No. 4,683,202
U.S. Pat. No. 4,751,177
U.S. Pat. No. 4,816,571
U.S. Pat. No. 4,868,105
U.S. Pat. No. 4,894,325
U.S. Pat. No. 4,959,463
U.S. Pat. No. 5,124,246
U.S. Pat. No. 5,141,813
U.S. Pat. No. 5,200,314
U.S. Pat. No. 5,214,136
U.S. Pat. No. 5,216,141
U.S. Pat. No. 5,223,618
U.S. Pat. No. 5,264,566
U.S. Pat. No. 5,273,882
U.S. Pat. No. 5,288,609
U.S. Pat. No. 5,378,825
U.S. Pat. No. 5,412,087
U.S. Pat. No. 5,428,148
U.S. Pat. No. 5,432,272
U.S. Pat. No. 5,445,934
U.S. Pat. No. 5,446,137
U.S. Pat. No. 5,457,025
U.S. Pat. No. 5,466,786
U.S. Pat. No. 5,470,967
U.S. Pat. No. 5,500,356
U.S. Pat. No. 5,539,082
U.S. Pat. No. 5,554,744
U.S. Pat. No. 5,574,146
U.S. Pat. No. 5,589,335
U.S. Pat. No. 5,602,240
U.S. Pat. No. 5,602,244
U.S. Pat. No. 5,610,289
U.S. Pat. No. 5,614,617
U.S. Pat. No. 5,623,070
U.S. Pat. No. 5,645,897
U.S. Pat. No. 5,652,099
U.S. Pat. No. 5,670,663
U.S. Pat. No. 5,672,697
U.S. Pat. No. 5,681,947
U.S. Pat. No. 5,700,922
U.S. Pat. No. 5,702,896
U.S. Pat. No. 5,708,154
U.S. Pat. No. 5,709,629
U.S. Pat. No. 5,714,324
U.S. Pat. No. 5,714,331
U.S. Pat. No. 5,714,606
U.S. Pat. No. 5,719,262
U.S. Pat. No. 5,723,597
U.S. Pat. No. 5,736,336
U.S. Pat. No. 5,744,305
U.S. Pat. No. 5,759,777
U.S. Pat. No. 5,763,167
U.S. Pat. No. 5,766,855
U.S. Pat. No. 5,773,571
U.S. Pat. No. 5,777,092
U.S. Pat. No. 5,786,461
U.S. Pat. No. 5,792,847
U.S. Pat. No. 5,858,988
U.S. Pat. No. 5,859,221
U.S. Pat. No. 5,872,232
U.S. Pat. No. 5,886,165
U.S. Pat. No. 5,891,625
U.S. Pat. No. 5,897,783
U.S. Pat. No. 5,908,845
U.S. Pat. No. 5,945,525
U.S. Pat. No. 6,001,983
U.S. Pat. No. 6,013,440
U.S. Pat. No. 6,037,120
U.S. Pat. No. 6,060,246
U.S. Pat. No. 6,090,548
U.S. Pat. No. 6,110,678
U.S. Pat. No. 6,140,496
U.S. Pat. No. 6,203,978
U.S. Pat. No. 6,221,581
U.S. Pat. No. 6,228,580
U.S. Pat. No. 6,309,823
U.S. Pat. No. 6,316,193
U.S. Pat. No. 6,322,971
U.S. Pat. No. 6,324,479
U.S. Pat. No. 6,329,140
U.S. Pat. No. 6,329,209
EP 266,032
PCT/EP/01219
PCT/US00/29865
WO 01/32672
WO 86/05815
WO90/06045
WO 92/20702
WO98/0914
The entire issue of Current Opinion in Microbiology, Volume 4, February 2001.
Amara et al., Nucl. Acids Res. 25:3465-3470, 1997.
Arfin et al., J. Biol. Chem. 275:29672-29684.
Ausubel et al., In: Current Protocols in Molecular Biology, John, Wiley & Sons, Inc, New York, 1994.
Beaucage, Methods Mol. Biol. 20:33-61, 1993.
Chuang et al., J. Bacteriol. 175:2026-2036, 1993.
Christensen, et al., J. PeptideSci. 3, 175-183, 1995.
Coombes et al., Infect. Immun. 69:1420-1427, 2001.
Cornelis et al., Curr. Opin. Microbiol. 4:13-15, 2001.
Cummings et al., Emerg. Inf. Dis. 6:513-524, 2000.
DeRisi et al., Nature Genetics 14:457-460, 1996.
Detweller et al., Proc. Natl. Acad. Sci. USA 98:5850-5855, 2001.
Dueholm et al., J. Org. Chem. 59, 5767-5773, 1994.
Egholm et al., Nature 365(6446):566-568, 1993.
Egholm et al., Nucleic Acids Res 23,217-222, 1995.
Feng et al., Proc. Natl. Acad. Sci. USA 97:6415-6420, 2000.
Fox, J. L. et al., ASM News 67:247-252, 2001.
Freier & Tinoco, Biochemistry 14, 3310-3314, 1975.
Froehler et al., Nucleic Acids Res., 14(13):5399-5407, 1986.
Gillam et al., J. Biol. Chem. 253(8):2532-9, 1978.
Gillam et al., Gene 8(1):99-106, 1979.
Gingeras et al., ASM News 66:463-469, 2000.
Graham et al., Curr. Opin. Microbiol. 4:65-70, 2001.
Graham et al., Proc. Natl. Acad. Sci. USA 96; 11554-11559, 1999.
Haaima, et al., 35, 1939-1942, Angew. Chem. Int. Ed. Engl. 1996
Ichikawa et al., Proc. Natl. Acad. Sci. USA 97:9659-9664, 2000.
Itakura et al., J. Am. Chem. Soc. 97(25):7327-32, 1975.
Kagnoff et al., Curr. Opin. Microbiol. 4:246-250, 2001.
Khorana, Science 203(4381):614-25, 1979.
Klug et al., Methods Enzymol. 152:316-325, 1987.
Koshlkin et al., Tetrahedron 54:3607-3630, 1998.
Koshlidn et al., J. Am. Chem. Soc. 120:13252-13253, 1998.
Kricka, Nonisotopic DNA Probe Techniques, Academic Press, San Diego, Calif., 1992.
Kumar et al., Bioorg. Med. Chem. Lett., 8:2219-2222, 1998.
Liang et al., Methods Enzymol. 254:304-321, 1995.
Lockhart et al., Nature Biotech. 14:1675, 1996.
Maskos et al., Nuc. Acids. Res. 20:1679-1684, 1992.
Merrifield, J. Am. Chem. Soc. 85:2149-2154, 1963.
Merrifield, Science, 232:341347, 1986.
Neidhardt et al., in Escherichia coli and Salmonella (Neidhardt, FC, Ed.), Vol. 1, pp. 13-16, ASM Press, Washington, D.C., 1996.
Newton et al., J. Comput. Biol. 8:37-52, 2001.
Norton et al., (Bioorg. Med. Chem. 3, 437-445, 1995.
Pietu et al., Genome Res. 6:492, 1996.
Plum, et al., Infect. Immun. 62:476-483, 1994.
Rappuoli, R. Proc. Natl. Acad. Sci. USA 97:13467-13469, 2000.
Robinson et al., Gene 148:137-141, 1994.
Rosenberger et al., J. Immunol. 164:5894-5904, 2000.
Sambrook et. al., In: Molecular Cloning: A Laboratory Manual, 2d Ed., Cold Spring Harbor Laboratory Press, Cold Spring Harbor, N.Y., 1989.
Sambrook et al., In: Molecular Cloning: A Laboratory Manual, 3rd Ed., Cold Spring Harbor Press, Cold Spring Harbor, N.Y., 2001.
Schena et al., Science 270:467-470, 1995a.
Schimmel et al., Biochemistry 11, 642-646, 1972.
Schena et al., Proc. Natl. Acad. Sci. USA 93:10539-11286, 1995b.
Shalon et al., Genome Res. 6:639-645, 1996.
Su et al., Molec. Biotechnol. 10:83-85, 1998.
Thomson et al., Tetrahedron 51, 6179-6194, 1995.
Uhlenbeck, J. Mol. Biol. 65, 25-41, 1972.
Velculescu et al., Science 270:484-487, 1995.
Wahlestedt et al., PNAS 97:5633-5638, 2000.
Wei et al., J. Bacteriol. 183:545-556, 2001.
Wendisch, et al., Anal. Biochem. 290:205-213, 2001.
Wood et al., Proc. Natl. Acad. Sci. USA. 82:1585-1588, 1985.
Yoshida et al., Nucl. Acids Res. 29:683-692, 2001.
Zhao et al., Gene 156:207, 1995.

1. A method of depleting hemoglobin mRNA in a hemoglobin mRNA-containing sample comprising:
binding a mixture of capture nucleic acids of SEQ ID NO:19-SEQ ID NO:28 to the sample in a reaction mixture; and
removing hemoglobin mRNA bound to the mixture of capture nucleic acids from the reaction mixture.

2. The method of claim 1, wherein the binding of the capture nucleic acids to the sample prevents amplification of the hemoglobin mRNA.

3-15. (canceled)

16. The method of claim 1, wherein said hemoglobin mRNA is a mammalian hemoglobin mRNA.

17. The method of claim 16, wherein said mammalian hemoglobin mRNA is a primate or murine hemoglobin mRNA.

18-25. (canceled)

26. The method of claim 1, wherein capture nucleic acids and hemoglobin mRNA are removed from the reaction mixture prior to amplification.

27. (canceled)

28. The method of claim 1, wherein the capture nucleic acids are attached to a solid surface prior to binding to the RNA.

29. The method of claim 1, wherein the capture nucleic acids are attached to a solid surface after binding to the RNA.

30. The method of claim 29, wherein the capture nucleic acids are attached to the solid surface by covalent binding.

31. The method of claim 29, wherein the capture nucleic acids are attached to die solid surface via a biotin/streptavidin system.

32. The method of claim 29, wherein the solid surface is a bead, a rod, or a plate.

33. The method of claim 32, wherein the solid surface is a bead and the bead comprises a super-paramagnetic material.

34. (canceled)

35. The method of claim 33, further comprising using a magnet to remove the bead from the reaction mixture prior to amplification.

36-76. (canceled)

77. A kit, in a suitable container, comprising a mixture of capture nucleic acids of SEQ ID NO:19-SEQ ID NO:28 and a super-paramagnetic bead.

78. The kit of claim 77 wherein said super-paramagnetic bead is coated by streptavidin and each of said capture nucleic acids comprises a biotin moiety.

79-84. (canceled)

85. The kit of claim 78, wherein each of said sequences SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28 is bound to a biotin moiety by a triethylene glycol linker.

86-90. (canceled)

91. A method of preventing poly(dT) primed reverse transcription of a target hemoglobin mRNA in a hemoglobin mRNA-containing sample comprising:
binding to the sample a first primer mix comprising SEQ ID NO:63-SEQ ID NO:73 that is specific to the target hemoglobin mRNA;
binding to the sample a second primer comprising a poly(dT) sequence; and
reverse transcribing the RNA in the hemoglobin mRNA-containing sample to form cDNA;
wherein the first primer mix prevents the reverse transcription of the target hemoglobin mRNA by the second primer.

92. (canceled)

93. The method of claim 91, further comprising extending the first primer mix to form a complementary DNA sequence prior to binding the second primer.

94. The method of claim 91, wherein the second primer comprises a RNA polymerase promoter sequence.

95. The method of claim 94, wherein the RNA polymerase promoter sequence is a T3 polymerase promoter sequence, a T7 polymerase promoter sequence, or a SP2 polymerase promoter sequence.

96-151. (canceled)

152. The method of claim 1 wherein the binding is in a reaction mixture comprising tetramethylammonium chloride or tetraethylammonium chloride.

153. The kit of claim 77 wherein the kit further comprises an isostabilizing agent.

154. The kit of claim 153 wherein the isostabilizing agent is tetramethylammonium chloride.
